(19) United States
(12) Patent Application Publication
(10) Pub. No.: US 2007/0037800 A1
(43) Pub. Date: Feb. 15, 2007

(54) Method of treating neurological disorders using clotrimazole and derivatives thereof

(75) Inventors: Christopher J. Cummings, San Francisco, CA (US); Gideon Shapiro, Gainesville, FL (US); Nagarajan S. Sankrithi, Ashland, MA (US); Richard Chesworth, Boston, MA (US)

Correspondence Address: EDWARDS & ANGELL, LLP, P.O. BOX 55874, BOSTON, MA, 02205, US

(73) Assignee: EnVivo Pharmaceuticals, Inc.

(21) Appl. No.: 11/473,479

(22) Filed: Jun. 23, 2006

Related U.S. Application Data
(60) Provisional application No. 60/694,025, filed on Jun. 23, 2005.

(51) Int. Cl. A61K 31/5375; A61K 31/4196; A61K 31/4192; A61K 31/415; A61K 31/41

(52) U.S. Cl. 514/231.2; 514/359; 514/381; 514/396; 514/406

(57) ABSTRACT

Methods and pharmaceutical compositions are disclosed for treating neurological disorders, such as Huntington's disease or Alzheimer's disease. The methods involve the administration of a triarylmethane compound, such as clotrimazole, or a salt thereof.

### CROSS-REFERENCE TO RELATED APPLICATIONS

This application claims the benefit of U.S. Provisional Application Ser. No. 60/694,025, filed Jun. 23, 2005, the contents of which are hereby incorporated by reference.

### FIELD OF THE INVENTION

The present invention relates generally to methods of treating neurological disorders, such as Huntington's disease or Alzheimer's disease, by the administration of clotrimazole or an analog or derivative thereof, such as tritylimidazoles and non-imidazole triphenylmethyl compounds, and its pharmaceutically acceptable salts. The invention also provides for a pharmaceutical composition for the treatment of a neurological disorder containing a tritylimidazole, such as clotrimazole, or a non-imidazole triphenylmethyl derivative, analog, or a salt thereof.

### BACKGROUND OF THE INVENTION

The clinical management of numerous neurological disorders has been frustrated by the progressive nature of degenerative, traumatic, or destructive neurological diseases and the limited efficacy and serious side-effects of available pharmacological agents. Conditions such as Huntington's disease, Alzheimer's disease, Parkinson's disease, severe seizure disorders (e.g., epilepsy and familial dysautonomia), as well as injury or trauma to the nervous system have eluded most conventional pharmacological attempts to alleviate or cure the conditions.

An exemplary neurological disorder is Huntington's disease which has proven particularly elusive to conventional pharmacological treatments. Huntington's disease (HD), a progressive hereditary disorder of the neurodegenerative type involving the basal ganglia (cerebral areas in charge of controlling involuntary movement), can cause highly debilitating motor and psychiatric symptoms. In most cases, onset of Huntington's disease occurs in the fertile age (around 35 to 40 years) with an incidence of one case in 10,000 and a mean duration of the disease of about 17 years. The onset is insidious and is characterized by abnormalities of coordination, movement, and behavior. Movement abnormalities include restlessness, mild postural abnormalities, and quick jerking movements of the fingers, limbs, and trunk. The movement abnormalities may be accompanied by substantial weight loss. Depression is common, and cognitive abnormalities and inappropriate behavior may develop. In contrast to the choreic movements typical of onset in adults, juvenile patients may exhibit rigidity, tremor, and dystonia. In the course of eight to 15 years, the disorder progresses to complete incapacitation, with most patients dying of aspiration pneumonia or inanition.

Huntington's disease was the first major inherited disorder with an unidentified basic defect to be linked with a DNA marker. Although knowledge of the underlying molecular basis for Huntington's disease has increased in recent years, pharmacological treatments based on this molecular knowledge have been limited to alleviating some of the symptoms associated with HD, a procedure that addresses neither the primary degenerative process nor the nonmotor aspects of the disease.

The genetic defect responsible for the disease consists of an expansion of the CAG triplet coding for the amino acid glutamine (polyQ expansion) at the amino-terminal end of the protein known as huntingtin. In healthy subjects, this triplet has a maximum number of repetitions of 36 glutamine residues; however in those affected, there is an increase in these repetitions ranging from about 38 to about 120 units. Within the scope of this variability, it has been observed that the greater the number of repetitions, the earlier the onset of the disease occurs. One hundred per cent of subjects with the mutation are affected, and the disease is transmitted with dominant autosomic characteristics; just one mutant allele is sufficient to evoke the pathology (Brinkman et al., Am J Hum Genet 60, 1202-1210 (1997)).

Methods such as cell transplantation have been of particular interest in the treatment of neurological diseases. However, mature neural tissues cannot be used for neural cell transplantation. Such tissues are not capable of surviving or establishing neurological function, which often depends on complex intercellular connections that cannot be surgically established. Thus, improved methods and compositions are needed for the effective treatment of neurological diseases.

### SUMMARY OF THE INVENTION

The present invention provides a method for treating a subject, such as an animal or human, having a neurological disorder.

In one aspect, the invention provides a method of treating a subject having a neurological disorder. The method includes the step of administering to said subject an effective amount of a compound of the Formula (I) or a pharmaceutically acceptable salt thereof:

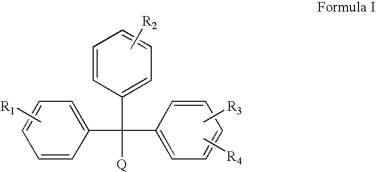

In Formula (I), R₁, R₂, R₃ and R₄ are independently selected from the group consisting of a hydrogen, halogen, cyano, trifluoromethyl, carboxylic acid (CO₂H), carboxamide (CON(R₅)₂), nitro, hydroxyl, alkoxy, mercapto, alkylthio, alkylsulfonyl, amino, alkylamino, dialkylamino, acylamino, aryl, heteroaryl, alkenyl, alkynyl, cycloalkyl, heterocycloalkyl, alkyl and substituted alkyl;
each R₅ is independently selected from the group consisting of a hydrogen, cycloalkyl, alkyl and substituted alkyl; and
Q is selected from the group consisting of a hydrogen, hydroxyl, alkoxy, alkylthio, alkylamino, dialkylamino, acylamino or a heterocyclic group.

In certain embodiments, the heterocyclic group is selected from the group consisting of N-morpholino, 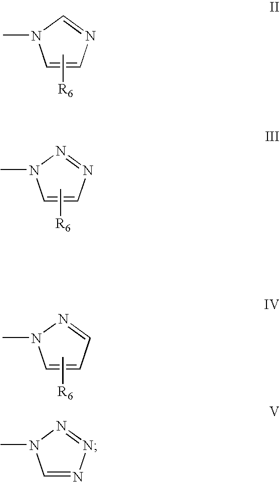
which R₆ is selected from the group consisting of a hydrogen, halogen, nitro, cyano, alkyl, alkoxy, and CON(R₅)₂.

In certain embodiments, the neurological disorder is a neurodegenerative disease. In certain embodiments, the neurological disorder is a disorder of movement. In certain embodiments, the neurological disorder is an extrapyramidal disorder or a cerebellar disorder. In certain embodiments, the neurological disorder is a hyperkinetic movement disorder. In certain embodiments, the neurological disorder is selected from the group consisting of Alzheimer's disease, Huntington's disease, Parkinson's disease, age-related memory impairment, amyotrophic lateral sclerosis, ataxia-telangiectasia, Biswanger's disease, cerebral amyloid angiopathies, Creutzfeldt-Jacob disease including variant form, corticobasal degeneration, multi infarct dementia, subcortical dementia, dementia with Lewy Bodies, dementia due to human immunodeficiency virus (HIV), Friedreich ataxia, fronto-temporal dementia linked to chromosome 17 (FTDP-17), frontotemporal lobar degeneration, frontal lobe dementia, Kennedy disease, Korsakoff's syndrome, mild cognitive impairment, neurological manifestations of HIV, neurological conditions arising from polyglutamine expansions, Pick's disease, prion diseases, Kuru disease, fatal familial insomnia, Gerstmann-Straussler-Scheinker disease, prion protein cerebral amyloid angiopathy, postencephalitic Parkinsonism, progressive supemuclear palsy, Rett syndrome, spinal muscular atrophy, transmissable spongiform encephalopathies and vascular dementia. In certain embodiments, the neurological disorder is selected from the group consisting of Alzheimer's disease, Huntington's disease, Parkinson's disease and a neurological condition arising from a polyglutamine expansion. In certain embodiments, the neurological disease is a neurological condition arising from a polyglutamine expansion. In certain embodiments, the polyglutamine expansion is of at least 10 residues. In certain embodiments, the polyglutamine expansion is of at least 20 residues. In certain embodiments, the polyglutamine expansion is between 21 and 33 residues in length. In certain embodiments, the neurological disorder is Huntington's disease.

In certain embodiments, the compound of the Formula (I) or a pharmaceutically acceptable salt thereof is administered in combination with at least one additional active agent. In certain embodiments, the additional active agent is selected from the group consisting of tiapride; pimozide; haloperidol; tetrabenazine; phenothiazines; an antiparkinsonian medication, such as levodopa, dopamine agonists, and anticholinergics; tricyclic antidepressants; SSRIs, monoamine oxidase inhibitors; benzodiazepines; amitriptyline; antipsychotics; propranolol; pindolo; classical antipsychotics; and clozapine.

In certain embodiments, the compound of the Formula (I) or a pharmaceutically acceptable salt thereof is administered as a pharmaceutical composition further comprising at least one excipient, carrier or diluent. In certain embodiments,the pharmaceutical composition is administered in a solid dosage form or in a liquid dosage form. In certain embodiments, the dosage form is selected from the group consisting of an oral dosage form, a parenteral dosage form, an intranasal dosage form, a suppository, a lozenge, a troche, buccal, a controlled release dosage form, a pulsed release dosage form, an immediate release dosage form, an intravenous solution, a suspension and combinations thereof. In certain embodiments, the dosage form is an oral dosage form. In certain embodiments, the oral dosage form is a controlled release dosage form. In certain embodiments, the oral dosage form is a tablet, capsule or a caplet. In certain embodiments, the pharmaceutical composition is administered using a shunt.

In certain embodiments, the subject is a mammal. In certain embodiments, the mammal is a human.

In certain embodiments, the compound of the Formula (I) is: 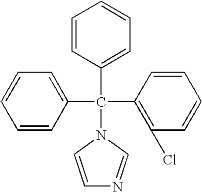
or a pharmaceutically acceptable salt thereof. In certain embodiments: the neurological disorder is a neurodegenerative disease; the neurological disorder is a disorder of movement; the neurological disorder is a neurodegenerative disease; the neurological disorder is a disorder of movement; the neurological disorder is an extrapyramidal disorder or a cerebellar disorder; the neurological disorder is a hyperkinetic movement disorder; the neurological disorder is selected from the group consisting of Alzheimer's disease, Huntington's disease, Parkinson's disease, age-related memory impairment, amyotrophic lateral sclerosis, ataxia-telangiectasia, Biswanger's disease, cerebral amyloid angiopathies, Creutzfeldt-Jacob disease including variant form, corticobasal degeneration, multi infarct dementia, subcortical dementia, dementia with Lewy Bodies, dementia due to human immunodeficiency virus (HIV), Friedreich ataxia, fronto-temporal dementia linked to chromosome 17 (FTDP-17), frontotemporal lobar degeneration, frontal lobe dementia, Kennedy disease, Korsakoff's syndrome, mild cognitive impairment, neurological manifestations of HIV, neurological conditions arising from polyglutamine expansions, Pick's disease, prion diseases, Kuru disease, fatal familial insomnia, Gerstmann-Straussler-Scheinker disease, prion protein cerebral amyloid angiopathy, postencephalitic Parkinsonism, progressive supemuclear palsy, Rett syndrome, spinal muscular atrophy, transmissable spongiform encephalopathies and vascular dementia; the neurological disorder is selected from the group consisting of Alzheimer's disease, Huntington's disease, Parkinson's disease and a neurological condition arising from a polyglutamine expansion; the neurological disease is a neurological condition arising from a polyglutamine expansion; the neurological disorder is an extrapyramidal disorder or a cerebellar disorder; the neurological disorder is a hyperkinetic movement disorder

In certain embodiments, the compound of the Formula (I) is: 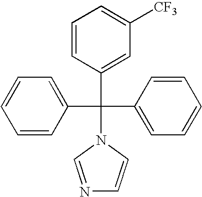
or a pharmaceutically acceptable salt thereof.

In certain embodiments, the compound of the Formula (I) is: 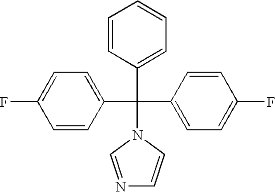
or a pharmaceutically acceptable salt thereof.

In certain embodiments, the compound of the Formula (I) is: 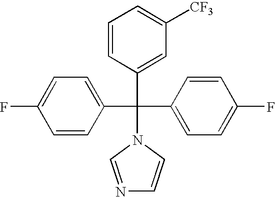
or a pharmaceutically acceptable salt thereof.

In another aspect, the invention provides a pharmaceutical composition for treating a subject having a neurological disorder. The pharmaceutical composition includes a pharmaceutically effective amount of a compound of the Formula (I) or a pharmaceutically acceptable salt thereof, and a pharmaceutically acceptable excipient, carrier or diluent.

In certain embodiments of the pharmaceutical composition, the compound of the Formula (I) is: 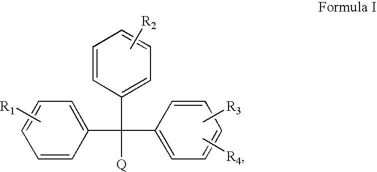
in which R₁, R₂, R₃ and R₄ are independently selected from the group consisting of a hydrogen, halogen, cyano, trifluoromethyl, carboxylic acid (CO₂H), carboxamide (CON(R₅)₂), nitro, hydroxyl, alkoxy, mercapto, alkylthio, alkylsulfonyl, amino, alkylamino, dialkylamino, acylamino, aryl, heteroaryl, alkenyl, alkynyl, cycloalkyl, heterocycloalkyl, alkyl and substituted alkyl;
each R₅ is independently selected from the group consisting of a hydrogen, cycloalkyl, alkyl and substituted alkyl; and
Q is selected from the group consisting of a hydrogen, hydroxyl, alkoxy, alkylthio, alkylamino, dialkylamino, acylamino or heterocyclic group.

In certain embodiments of the pharmaceutical composition, the heterocyclic group is selected from the group consisting of N-morpholino, 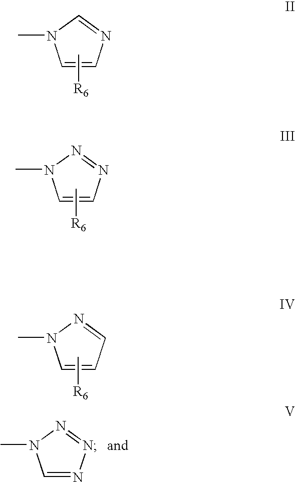
in which R₆ is selected from the group consisting of a hydrogen, halogen, nitro, cyano, alkyl, alkoxy, and CON(R₅)₂;
   or a pharmaceutically acceptable salt thereof.

In certain embodiments of the pharmaceutical composition, the compound of the Formula (I) is: 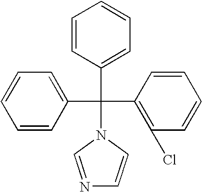
or a pharmaceutically acceptable salt thereof

In certain embodiments of the pharmaceutical composition, the compound of the Formula (I) is: 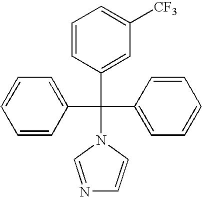
or a pharmaceutically acceptable salt thereof.

In certain embodiments of the pharmaceutical composition, the compound of the Formula (I) is: 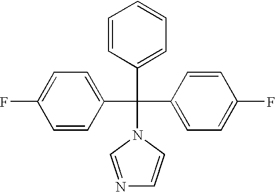
or a pharmaceutically acceptable salt thereof.

In certain embodiments of the pharmaceutical composition, the compound of the Formula (I) is: 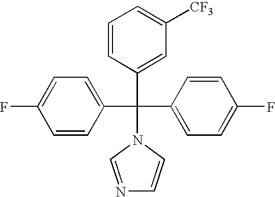
or a pharmaceutically acceptable salt thereof.

In another aspect, the invention provides compounds, e.g., any compounds or formula described herein. In certain embodiments, the compound is represented by the formula: 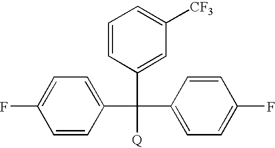
in which Q is selected from the group consisting of a hydrogen, hydroxyl, alkoxy, alkylthio, alkylamino, dialkylamino, acylamino or a heterocyclic group; or a pharmaceutically acceptable salt thereof.

In certain embodiments, the heterocyclic group, e.g., a nitrogen-containing heterocycle selected from the group consisting of N-morpholino, 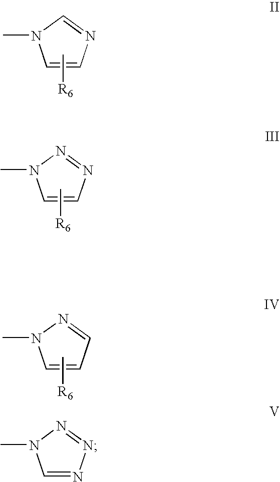
R₆ is selected from the group consisting of a hydrogen, halogen, nitro, cyano, alkyl, alkoxy, and CON(R₅)₂; and
each R₅ is independently selected from the group consisting of a hydrogen, cycloalkyl, alkyl and substituted alkyl.

In certain embodiments, the compound is represented by the structure: 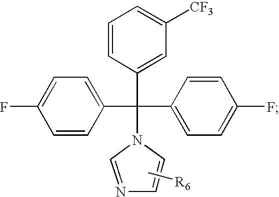
or a pharmaceutically acceptable salt thereof; in which R₆ is selected from the group consisting of a hydrogen, halogen, nitro, cyano, alkyl, alkoxy, and CON(R₅)₂; and each R₅ is independently selected from the group consisting of a hydrogen, cycloalkyl, alkyl and substituted alkyl.

In certain embodiments, the compound is: 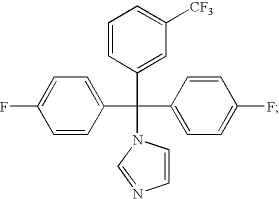
or a pharmaceutically acceptable salt thereof.

In another aspect, the invention provides a pharmaceutical composition. In certain embodiments, the pharmaceutical composition includes a compound represented by the formula: 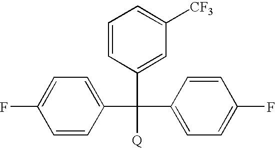
in which Q is selected from the group consisting of a hydrogen, hydroxyl, alkoxy, alkylthio, alkylamino, dialkylamino, acylamino or a heterocyclic group; or a pharmaceutically acceptable salt thereof;
   and a pharmaceutically acceptable excipient, carrier or diluent.

In another aspect, the invention provides method of treating a subject having a neurological disorder. The method includes administering to said subject an effective amount of a compound represented by the formula: 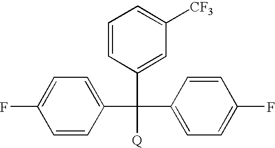
in which Q is selected from the group consisting of a hydrogen, hydroxyl, alkoxy, alkylthio, alkylamino, dialkylamino, acylamino or a heterocyclic group; or a pharmaceutically acceptable salt thereof.

### BRIEF DESCRIPTION OF THE DRAWINGS

FIG. 1 shows a comparison of the age-dependent decline in climbing speed between wild type flies (untreated) and HD model flies treated with clotrimazole (CLT; 50 and 100 μM), Trichostatin A (TSA; 250 μM), and carrier (1% DMSO) control flies, as described in Example 3.

FIG. 2 is a scatter plot depicting early climbing speed (1-7 days) plotted vs. late climbing speed (8-10 days) for several compounds of Formula (I), as described in Example 3. EVPK-0003546=CLT; EVPK-0004513=1-trimethylphenylimidazole.

FIG. 3 depicts a comparative survival plot for control flies (DMSO treated) vs. flies treated with Trichostatin A (TSA) and EVPK-0004523 (1-(3-(trifluoromethyl)trityl)imidazole), as described in Example 3.

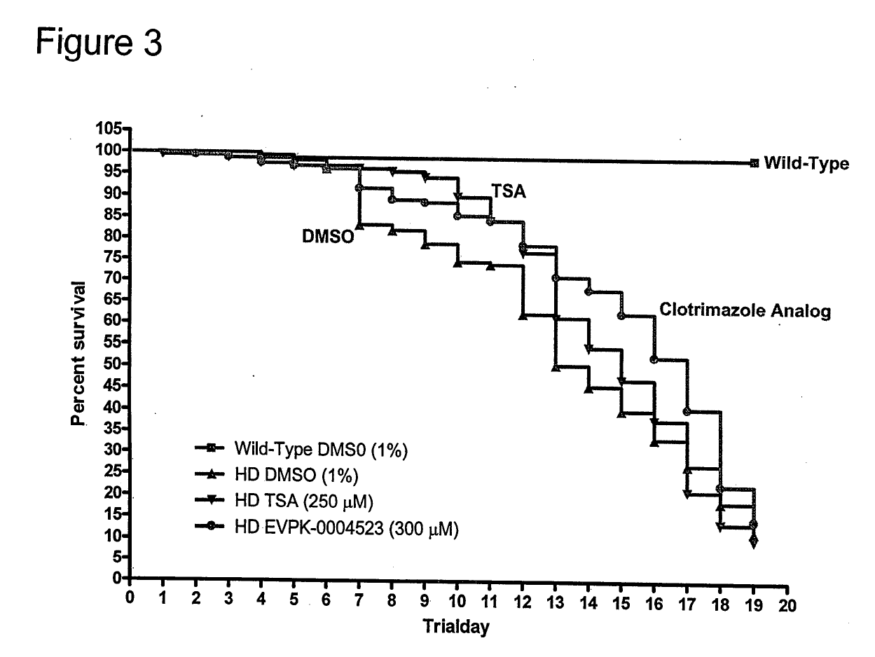

### DETAILED DESCRIPTION OF THE INVENTION

The present invention relates to compounds of Formula (I), and methods and pharmaceutical compositions for treating a subject, such as a human or an animal, that has a neurological disorder by administering a compound of Formula (I), or a derivative, analog, or a salt thereof. The invention further relates to pharmaceutical compositions and methods for treating a patient having a neurological disorder, comprising administering to the patient an effective amount of a compound of the Formula (I) or a pharmaceutically acceptable salt thereof.

Compounds of Formula (I) are represented by the structure:

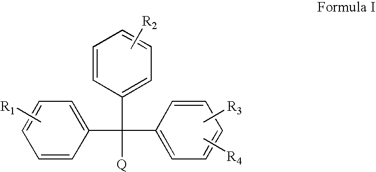

wherein R₁, R₂, R₃ and R₄ are independently selected from the group consisting of a hydrogen, halogen, cyano, trifluoromethyl, carboxylic acid (CO₂H), carboxamide (CON(R₅)₂), nitro, hydroxyl, alkoxy, mercapto, alkylthio, alkylsulfonyl, amino, alkylamino, dialkylamino, acylamino, aryl, heteroaryl, alkenyl, alkynyl, cycloalkyl, heterocycloalkyl and optionally substituted alkyl (e.g., unsubstituted or substituted alkyl);

wherein each R₅ is independently selected from the group consisting of a hydrogen, cycloalkyl, and alkyl group; and

wherein Q is selected from the group consisting of a hydrogen, hydroxyl, alkoxy, alkylthio, alkylamino, dialkylamino, N-morpholino, acylamino or heterocyclic group.

In certain embodiments, at least one of R₁ and R₂ is hydrogen. In certain embodiments, at least two of R₁, R₂, R₃ and R₄ are, independently, halogen, more preferably chloro or fluoro, and most preferably fluoro. In certain embodiments, when any of R₁, R₂, R₃ and R₄ are fluoro, the fluoro group is situated at the 4-position (para position) of the phenyl group to which it is attached. In certain embodiments, at least one of R₁, R₂, R₃ and R₄ is perfluoroalkyl, more preferably trifluoromethyl; in preferred embodiments, the perfluoroalkyl group is trifluoromethyl group at the 3-position (meta position) of the phenyl group to which it is attached.

In certain embodiments, the heterocyclic group is selected from the group consisting of N-morpholino,

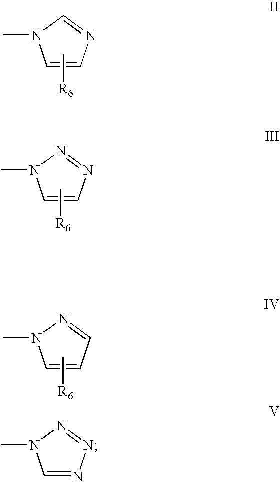

wherein R₆ is selected from the group consisting of a hydrogen, halogen, nitro, cyano, alkyl, alkoxy, and CON(R₅)₂. In certain embodiments, at least one of R₁ and R₂ is hydrogen.

As used herein, the term “alkyl” refers to a C₁ to C₆ straight or branched alkyl chain, which may be optionally substituted with one or more of the group consisting of a halogen, hydroxyl, alkoxy, amino, alkylamino, dialkylamino, N-morpholino, and carboxy, and combinations thereof.

The term “alkenyl,” as used herein, denote a monovalent group derived from a hydrocarbon moiety containing from two to six, or two to eight carbon atoms having at least one carbon-carbon double bond. Alkenyl groups include, but are not limited to, for example, ethenyl, propenyl, butenyl, 1-methyl-2-buten-1-yl, heptenyl, octenyl and the like.

The term “alkynyl,” as used herein, denote a monovalent group derived from a hydrocarbon moiety containing from two to six, or two to eight carbon atoms having at least one carbon-carbon triple bond. Representative alkynyl groups include, but are not limited to, for example, ethynyl, 1-propynyl, 1-butynyl, heptynyl, octynyl and the like.

As used herein, the term “aryl” refers to a mono- or bicyclic carbocyclic ring system having one or two aromatic rings including, but not limited to, phenyl, naphthyl, tetrahydronaphthyl, indanyl, idenyl and the like.

The term “heteroaryl,” as used herein, refers to a mono-, bi-, or tri-cyclic aromatic radical or ring having from five to ten ring atoms of which one ring atom is selected from S, O and N; zero, one or two ring atoms are additional heteroatoms independently selected from S, O and N; and the remaining ring atoms are carbon. Heteroaryl groups include, but are not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like.

As used herein, the term “halogen” refers to —F, —Cl, —Br, or —I.

As used herein, the term “perfluoroalkyl group” refers to an alkyl group in which all hydrogen atoms of the parent alkyl moiety have been replaced by fluorine atoms. Exemplary perfluoroalkyl groups include trifluoromethyl, pentafluoroethyl, octafluoropropyl, and the like.

As used herein, the term “cycloalkyl” refers to a C₃-C₁₀ (more preferably C₃-C₆) cyclic alkyl moiety, optionally substituted with one or more of the optional substituents described for alkyl groups, supra.

In one embodiment, the compound of Formula (I) can be: 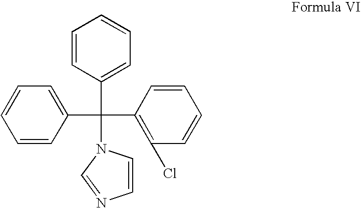
or a pharmaceutically acceptable salt thereof.

The compound of Formula VI is an exemplary tritylimidazole known as clotrimazole (CLT), a synthetic imidazole derivative. Clotrimazole is FDA-approved as an antifungal agent which is believed to act through inhibition of sterol-14-demethylase. Clotrimazole is available as an antifungal agent in several formulation, including lozenges, tablets, topical creams, and solutions. Oral CLT is also currently being investigated for treatment of sickle cell anemia, given its properties an inhibitor of the Gardos channel and subsequent effect in reducing red blood cell (RBC) dehydration.

In another embodiment, the compound of Formula (I) can be: 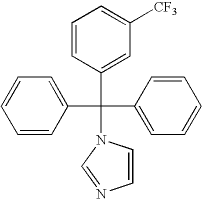
or a pharmaceutically acceptable salt thereof.

In yet another embodiment, the compound of Formula (I) can be: 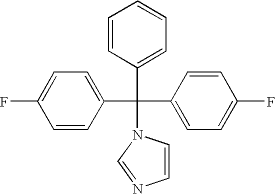
or a pharmaceutically acceptable salt thereof.

In yet another embodiment, the compound of Formula (I) can be: 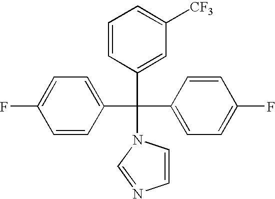
or a pharmaceutically acceptable salt thereof.

In yet a further embodiment, the compound of Formula (I) can be:

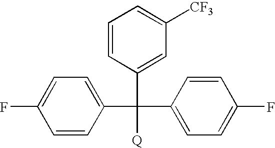

in which Q has the meaning of Formula (I), or a pharmaceutically acceptable salt thereof.

In certain embodiments, Q is a heterocyclic group, e.g., a nitrogen-containing heterocycle selected from the group consisting of N-morpholino, 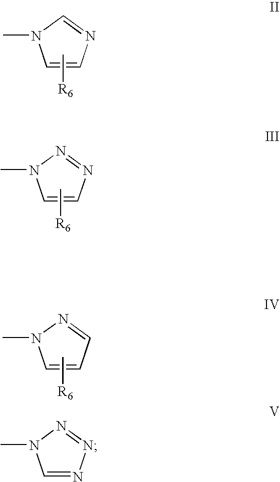
R₆ is selected from the group consisting of a hydrogen, halogen, nitro, cyano, alkyl, alkoxy, and CON(R₅)₂; and
each R₅ is independently selected from the group consisting of a hydrogen, cycloalkyl, alkyl and substituted alkyl.

In certain embodiments, the compound is represented by the structure: 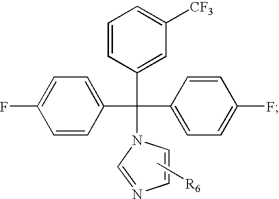
or a pharmaceutically acceptable salt thereof; in which R₆ is selected from the group consisting of a hydrogen, halogen, nitro, cyano, alkyl, alkoxy, and CON(R₅)₂; and each R₅ is independently selected from the group consisting of a hydrogen, cycloalkyl, alkyl and substituted alkyl.

In certain embodiments, the compound is: 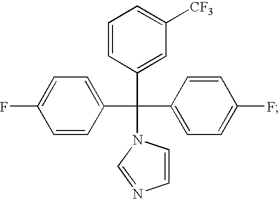
or a pharmaceutically acceptable salt thereof.
I. Definitions:

As used herein, the term “neural degeneration” means a condition in the central nervous system that gives rise to morphologic or developmental alteration of nervous or neurosensory organs, tissues, or cells; behavioral deficits; or locomotor deficits; wherein such alterations can be qualitatively or quantitatively analyzed in either larvae or adult flies.

As used herein, the term “candidate agent” refers to a biological or chemical compound that when administered to a transgenic fly has the potential to modify the phenotype of the fly, e.g. partial or complete reversion of the altered phenotype towards the phenotype of a wild type fly. “Agents” as used herein can include any recombinant, modified or natural nucleic acid molecule, library of recombinant, modified or natural nucleic acid molecules, synthetic, modified or natural peptide, library of synthetic, modified or natural peptides; and any organic or inorganic compound, including small molecules, or library of organic or inorganic compounds, including small molecules.

As used herein, the term “pharmaceutically acceptable salt” refers to those salts of the compounds formed by the process of the present invention which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid. Examples of pharmaceutically acceptable include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “small molecule” refers to compounds having a molecular mass of less than 3000 Daltons, preferably less than 2000 or 1500, more preferably less than 1000, and most preferably less than 600 Daltons. Preferably but not necessarily, a small molecule is a compound other than an oligopeptide.

As used herein, a “therapeutic agent” refers to a compound of Formula I that ameliorates one or more of the symptoms of a neurological disorder, including neurodegenerative disorders such as Huntington's and Alzheimer's disease in mammals, particularly humans. A therapeutic agent can reduce one or more symptoms of the disorder, delay onset of one or more symptoms, or prevent or cure the disease.

As used herein, an “additional active agent” refers to an agent that ameliorates one or more of the symptoms of a neurological disorder, including a neurodegenerative disorder such as Huntington's and Alzheimer's disease. An “additional active agent” can reduce one or more symptoms of the disorder, delay onset of one or more symptoms, or prevent or cure the disease. In the context of the present invention, an “additional active agent” can mean a second (different) therapeutic agent of the present invention (i.e., a different compound of Formula I), or a structurally distinct active agent for treating a neurological disorder.

An “effective amount” as referred to herein, relates to the amount of the compound of the Formula (I) that is capable of rendering a beneficial clinical outcome of the condition being treated with clotrimazole, or a derivative, anolog, or metabolite thereof compared with the absence of such treatment. The effective amount of the therapeutic agent administered will depend on the degree, severity, and type of the disease or condition, the amount of therapy desired, and the release characteristics of the pharmaceutical formulation. It will also depend on the subject's health, size, weight, age, sex and tolerance to specific compounds, which are determinable pharmaceutical parameters to those skilled in the field. Generally, treatment is considered “effective” if one or more symptoms of the disease or disorder improves (e.g., at least 10% relative to pre-treatment) during the course of treatment. The compounds of the invention can also be given to prevent or delay the onset of symptoms in an individual predisposed to such disorder, e.g., one predisposed to Alzheimer's or Huntington's disease. A delay or absence of the onset of symptoms relative to the time one would expect such symptoms to arise in a similar individual not treated with the drug would indicate efficacy.

As used herein, the term “transgenic fly” refers to a fly whose somatic and germ cells comprise a transgene operatively linked to a promoter, wherein the transgene encodes a human protein or polypeptide associated with a neurological disorder. For example, a transgenic fly useful for evaluating a compound of Formula I for treatment of a neurodegenerative disease such as Alzheimer's disease may comprise a Tau and/or human Aβ42_{Flemish} gene, wherein the expression of the transgenes in the nervous system results in the fly having a predisposition to, or resulting in, progressive neural degeneration. The term “double transgenic fly” refers to a transgenic fly whose somatic and germ cells comprise at least two transgenes, such as those that encode the Tau and human Aβ42_{Flemish}.

The terms “transgenic fly” and “double transgenic fly” include all developmental stages of the fly, i.e., embryonic, larval, pupal, and adult stages. The development of Drosophila is temperature dependent. The Drosophila egg is about half a millimeter long. It takes about one day after fertilization for the embryo to develop and hatch into a worm-like larva. The larva eats and grows continuously, molting one day, two days, and four days after hatching (first, second and third instars). After two days as a third instar larva, it molts one more time to form an immobile pupa. Over the next four days, the body is completely remodeled to give the adult winged form, which then hatches from the pupal case and is fertile after another day (timing of development is for 25° C.; at 18°, development takes twice as long).

As used herein, “fly” refers to an insect with wings, such as Drosophila. As used herein, the term “Drosophila” refers to any member of the Drosophilidae family, which include without limitation, Drosophila funebris, Drosophila multispina, Drosophila subfunebris, guttifera species group, Drosophila guttifera, Drosophila albomicans, Drosophila annulipes, Drosophila curviceps, Drosophila formosana, Drosophila hypocausta, Drosophila immigrans, Drosophila keplauana, Drosophila kohkoa, Drosophila nasuta, Drosophila neohypocausta, Drosophila niveifrons, Drosophila pallidiftons, Drosophila pulaua, Drosophila quadrilineata, Drosophila siamana, Drosophila sulfurigaster albostrigata, Drosophila sulfurigaster bilimbata, Drosophila sulfurigaster neonasuta, Drosophila Taxon F, Drosophila Taxon I, Drosophila ustulata, Drosophila melanica, Drosophila paramelanica, Drosophila tsigana, Drosophila daruma, Drosophila polychaeta, quinaria species group, Drosophila falleni, Drosophila nigromaculata, Drosophila palustris, Drosophila phalerata, Drosophila subpalustris, Drosophila eohydei, Drosophila hydei, Drosophila lacertosa, Drosophila robusta, Drosophila sordidula, Drosophila repletoides, Drosophila kanekoi, Drosophila virilis, Drosophila maculinatata, Drosophila ponera, Drosophila ananassae, Drosophila atripex, Drosophila bipectinata, Drosophila ercepeae, Drosophila malerkotliana malerkotliana, Drosophila malerkotliana pallens, Drosophila parabipectinata, Drosophila pseudoananassae pseudoananassae, Drosophila pseudoananassae nigrens, Drosophila varians, Drosophila elegans, Drosophila gunungcola, Drosophila eugracilis, Drosophila ficusphila, Drosophila erecta, Drosophila mauritiana, Drosophila melanogaster, Drosophila orena, Drosophila sechellia, Drosophila simulans, Drosophila teissieri, Drosophila yakuba, Drosophila auraria, Drosophila baimaii, Drosophila barbarae, Drosophila biauraria, Drosophila birchii, Drosophila bocki, Drosophila bocqueti, Drosophila burlai, Drosophila constricta (sensu Chen & Okada), Drosophila jambulina, Drosophila khaoyana, Drosophila kikkawai, Drosophila lacteicornis, Drosophila leontia, Drosophila lini, Drosophila mayri, Drosophila parvula, Drosophila pectinifera, Drosophila punjabiensis, Drosophila quadraria, Drosophila rufa, Drosophila seguyi, Drosophila serrata, Drosophila subauraria, Drosophila tani, Drosophila trapezifrons, Drosophila triauraria, Drosophila truncata, Drosophila vulcana, Drosophila watanabei, Drosophila fuyamai, Drosophila biarmipes, Drosophila mimetica, Drosophila pulchrella, Drosophila suzukii, Drosophila unipectinata, Drosophila lutescens, Drosophila paralutea, Drosophila prostipennis, Drosophila takahashii, Drosophila trilutea, Drosophila bifasciata, Drosophila imaii, Drosophila pseudoobscura, Drosophila saltans, Drosophila sturtevanti, Drosophila nebulosa, Drosophila paulistorum, and Drosophila willistoni. In one embodiment, the fly is Drosophila melanogaster.

As used herein, the term “phenotype” As used herein, the term “phenotype” with respect to a transgenic fly refers to an observable and/or measurable physical, behavioral, or biochemical characteristic of a fly. The term “altered phenotype” or “change in phenotype” as used herein, refers to a phenotype that has changed measurably or observably relative to the phenotype of a wild-type fly. Examples of altered phenotypes include behavioral phenotypes, such as appetite, mating behavior, and/or life span; morphological phenotypes, such as rough eye phenotype, concave wing phenotype, or any different shape, size, color, growth rate or location of an organ or appendage, or different distribution, and/or characteristic of a tissue or cell, as compared to the similar characteristic observed in a control fly; and locomotor dysfinction phenotypes, such as reduced climbing ability, reduced walking ability, reduced flying ability, decreased speed or acceleration, abnormal trajectory, abnormal turning, and abnormal grooming. An altered phenotype is a phenotype that has changed by a measurable amount, e.g., by at least a statistically significant amount, preferably by at least 1%, 5%, 10%, 20%, 30%, 40%, or 50% relative to the phenotype of a control fly. As used herein, “a synergistic altered phenotype” or “synergistic phenotype,” refers to a phenotype wherein a measurable and/or observable physical, behavioral, or biochemical characteristic of a fly is more than the sum of its components.

As used herein, the “rough eye” phenotype is characterized by irregular ommatidial packing, occasional ommatidial fusions, and missing bristles that can be caused by degeneration of neuronal cells. The eye becomes rough in texture relative to its appearance in wild type flies, and can be easily observed by microscope. Neurodegeneration is readily observed and quantified in a fly's compound eye, which can be scored without any preparation of the specimens (Fernandez-Funez et al., 2000, Nature 408:101-106; Steffan et. al, 2001, Nature 413:739-743; Agrawal et al., 2005, Proc. Natl. Acad. Sci. USA 102:3777-3781). This organism's eye is composed of a regular trapezoidal arrangement of seven visible rhabdomeres produced by the photoreceptor neurons of each Drosophila ommatidium. Expression of mutant transgenes specifically in the Drosophila eye leads to a progressive loss of rhabdomeres and subsequently a rough-textured eye, which can be expressed quantitatively, for example, as the number of rhabdomeres per ommatidium (Fernandez-Funez et al., 2000; Steffan et. al, 2001). Administration of therapeutic compounds to these organisms slows the photoreceptor degeneration and improves the rough-eye phenotype (Steffan et. al, 2001).

As used herein, the “concave wing” phenotype is characterized by abnormal folding of the fly wing such that wings are bent upwards along their long margins.

As used herein, “locomotor dysfunction” refers to As used herein, “locomotor dysfunction” refers to a phenotype where flies have a deficit in motor activity, movement, or response to a stimulus (e.g., at least a statistically significant difference, or at least a 10% difference in a measurable parameter) as compared to control flies. Motor activities include flying, climbing, crawling, and turning. In addition, movement traits where a deficit can be measured include, but are not limited to: i) average total distance traveled over a defined period of time; ii) average distance traveled in one direction over a defined period of time; iii) average speed (average total distance moved per time unit); iv) distance moved in one direction per time unit; v) acceleration (the rate of change of velocity with respect to time; vi) turning; vii) stumbling; viii) spatial position of a fly to a particular defined area or point; ix) path shape of the moving fly; and x) undulations during larval movement; xi) rearing or raising of larval head; and xii) larval tail flick. Examples of movement traits characterized by spatial position include, without limitation: (1) average time spent within a zone of interest (e.g., time spent in bottom, center, or top of a container; number of visits to a defined zone within container); and (2) average distance between a fly and a point of interest (e.g., the center of a zone). Examples of path shape traits include the following: (1) angular velocity (average speed of change in direction of movement); (2) turning (angle between the movement vectors of two consecutive sample intervals); (3) frequency of turning (average amount of turning per unit of time); and (4) stumbling or meander (change in direction of movement relative to the distance). Turning parameters can include smooth movements in turning (as defined by small degrees rotated) and/or rough movements in turning (as defined by large degrees rotated). Locomotor phenotypes can be analyzed using methods described, for example, in U.S. Application Nos. 2004/0076583, 2004/0076318, and 2004/0076999, each of which is hereby incoporated by reference in its entirety.

A phenoprofile of a test or reference population is determined by measuring traits of the population. The present invention allows simultaneous measurement of multiple traits of a population. Although a single trait may be measured, multiple traits can also be measured. For example, at least 2, at least 3, at least 4, at least 5, at least 7 or at least 10 traits can be assessed for a population. The traits measured can be solely movement traits, solely behavioral traits solely morphological traits or a mixture of traits in multiple categories. In some embodiments at least one movement trait and at least one non-movement trait are assessed.

As used herein, a “control fly” or “wild type fly” refers to a larval or adult fly of the same genotype of the transgenic fly as to which it is compared, except that the control fly either i) does not comprise the transgene(s) present in the transgenic fly, or ii) has not been administered a candidate agent.

II. Compounds and Synthesis

The compounds of the invention can be prepared by a variety of methods, some of which are known in the art. Appropriate starting materials and reagents can be obtained commercially or can be prepared by standard procedures. Examples of syntheses are provided in the Examples (see below).

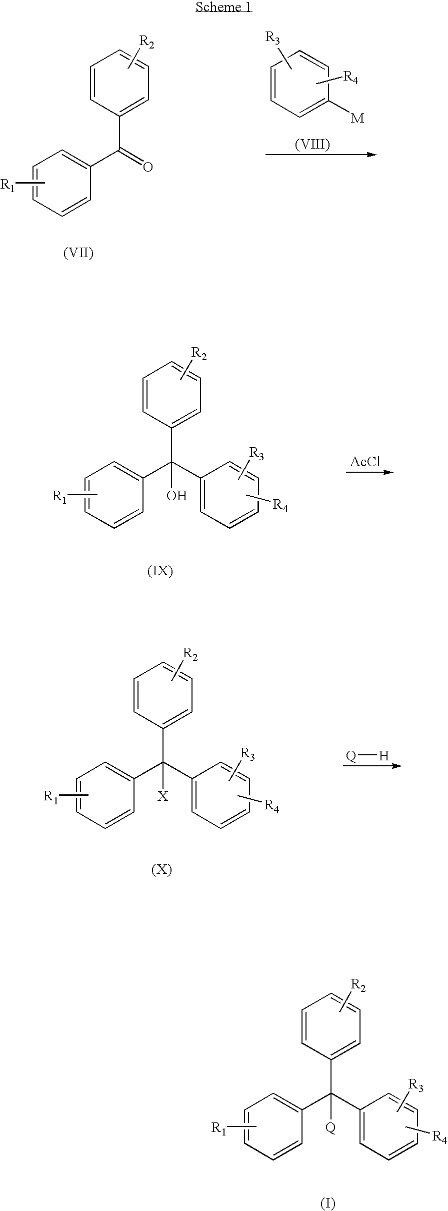

Schemes 1-4 illustrate exemplary synthetic routes to compounds of the invention, in which R₁-R₄ represent optional substituents. Benzophenones of Formula (VII) (Scheme 1) can be reacted with a metallated aromatic compound of Formula (VIII), where M represents a suitable metal, with appropriate ligands, such as lithium or magnesium-halide. The metallation reaction can be performed in a non-protic solvent such as THF, DME, MTBE, Et₂O, and the like, or a combination of such solvents. The metallation reaction is usually carried out at a temperature between −78 and 80° C., preferably at −20° C. to room temperature. For example, when M=MgBr (Grignard reagent), the metallated species can be generated by treatment of the corresponding aryl bromide with metallic magnesium, or if M=Li, the metallated species can be generated by treating the corresponding aryl bromide with butyl lithium at −78° C. Alternatively, certain metallated aryl compounds of Formula (VIII) can be purchased commercially.

Upon reaction of the metalled aryl compound with the benzophenone, the resultant triaryl alcohols of Formula (IX) can be converted into the corresponding halides of Formula (X), where X represents a halogen atom, preferably chlorine. The transformation may be performed, as shown in Scheme 1, by treating the alcohol of Formula (IX) with a reagent such as acetyl chloride (e.g., for X=Cl) in a non-protic solvent such as THF, CH₂Cl₂, CHCl₃ or Et₂O (CH₂Cl₂ is preferred). The reaction is advantageously performed at room temperature with optional cooling.

The halogenated triaryl compound of Formula (X) can then be converted into a product of Formula (I), where Q represents alkoxy, alkylthio, alkylamino, di-alkylamino, N-morpholino or a heterocyclic group. The transformation is generally achieved by treating the halo compound (X) with a compound of formula Q-H in the presence of a base such as K₂CO₃, NMO, Et₃N, EtNⁱPr₂ in a solvent such as MeCN, CH₂Cl₂ or THF. When the moiety Q contains a nucleophilic nitrogen atom, the solvent is preferably MeCN and the base is preferably Et₃N.

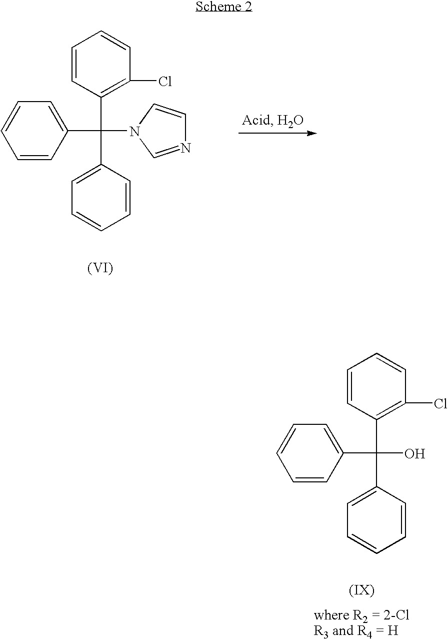

When an alcohol of Formula (IX) has R₁=2-Cl and R₂, R₃ and R₄ are all hydrogen, the compound can be prepared directly from clotrimazole (VI), as shown in Scheme 2. The transformation can be effected by heating clotrimazole (VI) in the presence of water and a strong acid such as hydrochloric acid, sulfuric acid, MsOH, p-TsOH or nitric acid. Aqueous HCl is preferred.

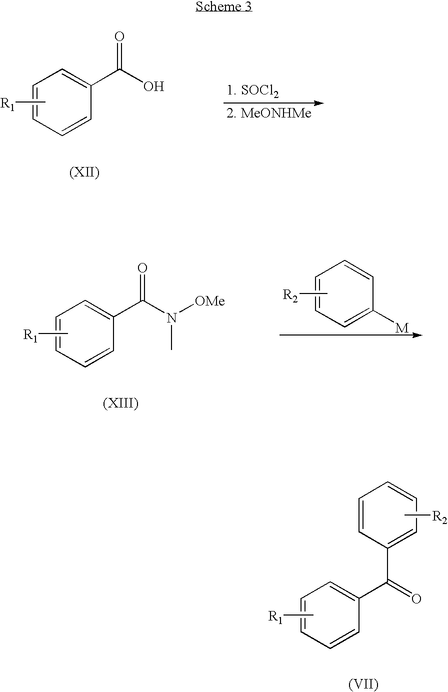

Benzophenones of Formula (VII) may be commercially available. Benzophenones can alternatively be prepared by methods known to those of ordinary skill in the art. An example of such a method is depicted in Scheme 3, in which an N-methyl-N-methoxy benzamide of Formula (VIII) is reacted with a reactive metallated phenyl species (e.g. phenyl lithium or phenyl-Grignard reagent, which may optionally be substituted) to yield a benzophenone of Formula (I). The amides of Formula (VIII) may be prepared by Weinreb amidation, e.g., from the corresponding benzoic acid chloride and (Me)OMe (N,O-dimethylhydroxylamine). The acid chloride may be prepared from the corresponding carboxylic acid (VII). Conversion of a carboxylic acid to an acid chloride is well known to those of ordinary skill in the art, e.g. by heating the carboxylic acid with thionyl chloride or treating the carboxylic acid with oxalyl chloride with a catalytic amount of DMF. Alternatively, the amide (VIII) can be prepared directly from the carboxylic acid using a coupling agent and HN(Me)OMe. Suitable coupling agents are well known and include (without limitaion)N-cyclohexyl-N′-(4-diethylaminocyclohexyl)-carbodiimide (DCC), 1-(3-dimethylaminopropyl)-3-ethylcarbodiimide (EDC) and bromotripyrrolidino phosphonium hexafluorophosphate (PyBroP®), benzotriazole1-1yl-oxy-tris-pyrrolidino phosphonium hexafluorophosphate (PyBOP®), with suitable additives, if necessary, include 1-hydroxybenzotriazole (HOBt) and 3-hydroxy-4-oxo-3,4-dihydro-1,2,3-benzotriazine.

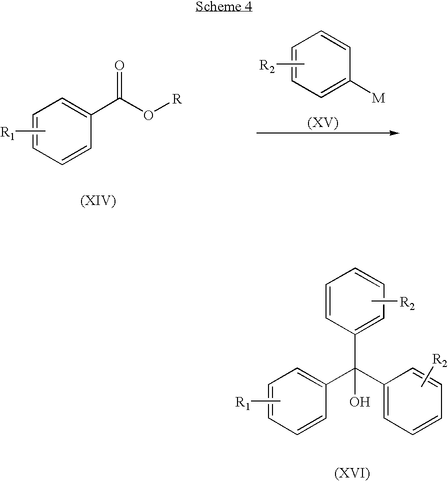

When at least two of the three aryl groups of Formula (IX) are equivalent, the synthesis of a triaryl alcohol of Formula (XVI) may be accomplished as depicted in Scheme 4. A benzoic ester of Formula (XIV), in which R represents alkyl (more preferably methyl or ethyl), can be treated with at least two equivalents of a metallated aromatic of Formula (XV), where M represents a suitable metal with appropriate ligands such as lithium or a magnesium-halide (e.g. Grignard reagent). The reaction is performed in a non-protic solvent such as THF, DME, MTBE, Et₂O or a combination of the said solvents. The reaction is usually carried at a temperature between −78 and 80° C., preferably at −20° C. to room temperature.

It will be appreciated that the order of certain steps in the above schemes may be altered. Moreover, reactive groups not involved in the above processes can be protected with standard protecting groups during the reactions; protective groups can be removed by standard procedures known to those of ordinary skill in the art (see, e.g., T. W. Greene & P. G. M. Wuts, Protecting Groups in Organic Synthesis, Third Edition, 1999, Wiley-Interscience). Examples of protecting groups include methyl, benzyl, acetate and tetrahydropyranyl for the hydroxyl moiety, and BOC, CBz, trifluoroacetamide and benzyl for the amino moiety, methyl, ethyl, tert-butyl and benzyl esters for the carboxylic acid moiety.

The following references also contain data and procedures relevant to the synthesis of the compounds described and are incorporated herein by reference in their entireties.

### REFERENCES

1. K. H. Buechel, E. Regel, M. S. Plempel DE 19670915.
2. H. Wulff, M. J. Miller, W. Hansel, S. Grissmer, M. D. Cahalan and K. G. Chandy Proc. Natl. Acad. Sciences 2000, 97, 8151-56.
3. K. H. Buechel, W. Draber and M. Plempel Arzneim.-Forsch (Drug Res.) 1972, 22 (8), 1260-1272.
4. E. M. Arnett, R. A. Flowers II, R. T. Ludwig, A. E. Meekhof and S. A. Walek J. Phys. Org. Chem. 1997, 10, 499-513
5. R. A. Al-Qawasmeh, Y. Lee, M-Y. Cao, X. Gu, A. Vassilakos, J. A. Wright and A. Young. Bioorganic and Med. Chem. Lett. 2004, 14, 347-350.
   III. Neurological Disorders

The neurological disorder treated in the method of the invention can be, but is not limited to, a disorder of movement, an extrapyramidal disorder, a cerebellar disorder, or a hyperkinetic movement disorder. The neurological disorder also can be, but is not limited to, Alzheimer's disease, Huntington's disease, Parkinson's disease, age-related memory impairment, amyotrophic lateral sclerosis, ataxia-telangiectasia, Biswanger's disease, cerebral amyloid angiopathies, Creutzfeldt-Jacob disease including variant form, corticobasal degeneration, multi infarct dementia, subcortical dementia, dementia with Lewy Bodies, dementia due to human immunodeficiency virus (HIV), Friedreich ataxia, fronto-temporal dementia linked to chromosome 17 (FTDP-17), frontotemporal lobar degeneration, frontal lobe dementia, Kennedy disease, Korsakoff's syndrome, mild cognitive impairment, neurological manifestations of HIV, neurological conditions arising from polyglutamine expansions, Pick's disease, prion diseases, Kuru disease, fatal familial insomnia, Gerstmann-Straussler-Scheinker disease, prion protein cerebral amyloid angiopathy, postencephalitic Parkinsonism, progressive supemuclear palsy, spinal muscular atrophy, transmissable spongiform encephalopathies or vascular dementia. The present inventive method also can provide therapeutic benefit to diseases or conditions including, but is not limited to, agyrophilic grain dementia, Parkinsonism-dementia complex of Guam, auto-immune conditions such as Guillain-Barre syndrome or Lupus, brain and spinal tumors (including neurofibromatosis), cerebral amyloid angiopathies, cerebral palsy, chronic fatigue syndrome, corticobasal degeneration, conditions due to developmental dysfunction of the CNS parenchyma, conditions due to developmental dysfunction of the cerebrovasculature, dementia lacking distinct histology, Dementia Pugilistica, diffuse neurofibrillary tangles with calcification, diseases of the eye, ear and vestibular systems involving neurodegeneration (including macular degeneration and glaucoma), Down's syndrome, dyskinesias (Paroxysmal), dystonias, essential tremor, Fahr's syndrome, hepatic encephalopathy, hereditary spastic paraplegia, hydrocephalus, pseudotumor cerebri and other conditions involving CSF dysfunction, Gaucher's disease, Hallervorden-Spatz disease, Korsakoff's syndrome, mild cognitive impairment, monomelic amyotrophy, motor neuron diseases, multiple system atrophy, multiple sclerosis and other demyelinating conditions (e.g., leukodystrophies), myalgic encephalomyelitis, myoclonus, neurodegeneration induced by chemicals, drugs and toxins, neurological/cognitive manifestations and consequences of bacterial and/or viral infections, including but not restricted to enteroviruses, Niemann-Pick disease, non-Guamanian motor neuron disease with neurofibrillary tangles, non-ketotic hyperglycinemia, olivo-ponto cerebellar atrophy, oculopharyngeal muscular dystrophy, neurological manifestations of Polio myelitis including non-paralytic polio and post-polio-syndrome, primary lateral sclerosis, restless leg syndrome, Sandhoff disease, spasticity, sporadic fronto-temporal dementias, striatonigral degeneration, subacute sclerosing panencephalitis, sulphite oxidase deficiency, Sydenham's chorea, tangle only dementia, Tay-Sach's disease, Tourette's syndrome, vascular dementia, and Wilson disease.

In one embodiment of the invention, the neurological disease is a neurological condition arising from a polyglutamine expansion. The polyglutamine expansion can be of at least about 10, at least about 20, at least about 30, at least about 40, at least about 50, at least about 60, at least about 70, at least about 80, or at least about 100 or more residues. In Huntington's disease, for example, the polyglutamine expansion is typically between 21 and 33 residues in length.

The subject that the method of treatment is administered to can be an animal, such as Drosophila or a mammal. The mammal can be, but is not limited to, a mouse, a rat, a cat, a dog, a primate, or a human.

III. Animal Models:

The present invention discloses methods and pharmaceutical compositions for treating neurological disorders, such as neurodegenerative diseases, comprising a compound of the Formula I (above). The suitability of a compound for treatment of a neurodegenerative disease can be assessed in any of a number of animal models for neurodegenerative disease. For example, mice transgenic for an expanded polyglutamine repeat mutant of ataxin-1 develop ataxia typical of spinocerebellar ataxia type 1 (SCA-1) are known (Burright et al., 1995, Cell 82: 937-948; Lorenzetti et al., 2000, Hum. Mol. Genet. 9: 779-785; Watase, 2002, Neuron 34: 905-919), and can be used to determine the efficacy of a given compound in the treatment or prevention of neurodegenerative disease. Additional animal models, for example, for Huntington's disease (see, e.g., Mangiarini et al., 1996, Cell 87: 493-506, Lin et al., 2001, Hum. Mol. Genet. 10: 137-144), Alzheimer's disease (Hsiao, 1998, Exp. Gerontol. 33: 883-889; Hsiao et al., 1996, Science 274: 99-102), Parkinson's disease (Kim et al., 2002, Nature 418: 50-56), amyotrophic lateral sclerosis (Zhu et al., 2002, Nature 417: 74-78), Pick's disease (Lee & Trojanowski, 2001, Neurology 56 (Suppl. 4): S26-S30, and spongiform encephalopathies (He et al., 2003, Science 299: 710-712) can be used to evaluate the efficacy of the compounds of the Formula (I) in a similar manner.

Animal models are not limited to mammalian models. For example, Drosophila strains provide accepted models for a number of neurodegenerative disorders (reviewed in Fortini & Bonini, 2000, Trends Genet. 16: 161-167; Zoghbi & Botas, 2002, Trends Genet. 18: 463-471). These models include not only flies bearing mutated fly genes, but also flies bearing human transgenes, optionally with targeted mutations. Among the Drosophila models available are, for example, spinocerebellar ataxias (e.g., SCA-1 (see, e.g., WO 02/058626), SCA-3 (Warrick et al., 1998, Cell 93: 939-949)), Huntington's disease (Kazemi-Esfaijani & Benzer, 2000, Science 287: 1837-1840), Parkinson's disease (Feany et al., 2000, Nature 404: 394-398; Auluck et al., 2002, Science 295: 809-810), age-dependent neurodegeneration (Palladino et al., 2002, Genetics 161: 1197-1208), Alzheimer's disease (Selkoe et al., 1998, Trends Cell Biol. 8: 447-453; Ye et al., 1999, J. Cell Biol. 146: 1351-1364), amyotrophic lateral sclerosis (Parkes et al., 1998, Nature Genet. 19: 171-174), and adrenoleukodystrophy.

The use of Drosophila as a model organism has proven to be an important tool in the elucidation of human neurodegenerative pathways, as the Drosophila genome contains many relevant human orthologs that are extremely well conserved in function (Rubin, G.M., et al., Science 287: 2204-2215 (2000)). For example, Drosophila melanogaster carries a gene that is homologous to human APP which is involved in nervous system function. The gene, APP-like (APPL), is approximately 40% identical to APP695, the neuronal isoform (Rosen et al., Proc. Natl. Acad. Sci. U.S.A. 86:2478-2482 (1988)), and like human APP695 is exclusively expressed in the nervous system. Flies deficient for the APPL gene show behavioral defects which can be rescued by the human APP gene, suggesting that the two genes have similar functions in the two organisms (Luo et al., Neuron 9:595-605 (1992)). In addition, Drosophila models of polyglutamine repeat diseases (Jackson, G. R., et al., Neuron 21:633-642 (1998); Kazemi-Esfarani, P. and Benzer, S., Science 287:1837-1840 (2000); Femandez-Funez et al., Nature 408:101-6 (2000)), Parkinson's disease (Feany, M. B. and Bender, W. W., Nature 404:394-398 (2000)) and other diseases have been established which closely mimic the disease state in humans at the cellular and physiological levels, and have been successfully employed in identifying other genes that may be involved in these diseases. Thus, the power of Drosophila as a model system has been demonstrated in the ability to represent the disease state and to perform large scale genetic screens to identify critical components of disease.

The transgenic flies exhibit progressive neurodegeneration which can lead to a variety of altered phenotypes including locomotor phenotypes, behavioral phenotypes (e.g., appetite, mating behavior, and/or life span), and morphological phenotypes (e.g., shape, size, or location of a cell, organ, or appendage; or size, shape, or growth rate of the fly).

Test animals, such as transgenic flies, are administered a compound of Formula (I) and evaluated for symptoms relative to animals not administered the compound. A change in the severity of symptoms (e.g., a 1%, 2%, 5%, 10%, or greater improvement in one or more symptoms), or a delay in the onset of symptoms, in treated versus untreated animals can be indicative of therapeutic efficacy.

For example, clotrimazole (CLT) has shown reproducible positive effects in a Drosophila model of HD. The flies performed better than carrier controls using two metrics: early speed (days 1-7) and late speed (days 8-10). One close CLT analog, 1-(3-(trifluoromethyl)trityl)imidazole hydrochloride, also had therapeutic effect in the same Drosophila HD model. This is the first evidence of CLT being neuroprotective in a polyglutamine-induced neurotoxicity model.

IV. Dosage and Administration

The present invention is also drawn to a pharmaceutical composition for treating a subject having a neurological disorder comprising a therapeutically effective amount of a compound of the Formula (I), a derivative or a pharmaceutically acceptable salt thereof, and a pharmaceutically acceptable excipient, carrier or diluent. The pharmaceutical composition can comprise, but is not limited to, clotrimazole or trifluoromethyl-tritylimidazole, or a derivative, analog, metabolite or a pharmaceutically acceptable salt thereof.

The pharmaceutical composition of the method of the present invention can be administered in a variety of dosage forms including, but not limited to, a solid dosage form or in a liquid dosage form, an oral dosage form, a parenteral dosage form, an intranasal dosage form, a suppository, a lozenge, a troche, buccal, a controlled release dosage form, a pulsed release dosage form, an immediate release dosage form, an intravenous solution, a suspension or combinations thereof. An oral dosage form is preferred. The dosage can be an oral dosage form that is a controlled release dosage form. The oral dosage form can be a tablet, a capsule, or a caplet. The compounds employed in the present invention can be administered, for example, by oral or parenteral routes, including intravenous, intramuscular, intraperitoneal, subcutaneous, transdermal, airway (aerosol), rectal, vaginal and topical (including buccal and sublingual) administration. In one embodiment, the compounds or pharmaceutical compositions comprising the compounds are delivered to a desired site, such as the brain, by continuous injection via a shunt.

In another embodiment, the compound of Formula (I) of the inventive method can be administered parenterally, such as intravenous (IV) administration. The formulations for administration will commonly comprise a solution of the compound of the Formula (I) (e.g., clotrimazole) dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of compound of Formula (I) in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In one embodiment, the compound of Formula (I) of the inventive method can be administered by introduction into the central nervous system of the subject, e.g., into the cerbrospinal fluid of the subject. The formulations for administration will commonly comprise a solution of the compound of the Formula (I) (e.g., clotrimazole) dissolved in a pharmaceutically acceptable carrier. In certain aspects of the invention, the compound of the Formula (I) is introduced intrathecally, e.g., into a cerebral ventricle, the lumbar area, or the cistema magna. In another aspect, the compound of the Formula (I) is introduced intraocullarly, to thereby contact retinal ganglion cells.

The pharmaceutically acceptable formulations can easily be suspended in aqueous vehicles and introduced through conventional hypodermic needles or using infusion pumps. Prior to introduction, the formulations can be sterilized with, preferably, gamma radiation or electron beam sterilization, described in U.S. Pat. No. 436,742 the contents of which are incorporated herein by reference.

In one embodiment, the pharmaceutical composition comprising a compound of Formula (I) is administered into a subject intrathecally. As used herein, the term “intrathecal administration” is intended to include delivering a pharmaceutical composition comprising a compound of Formula (I) directly into the cerebrospinal fluid of a subject, by techniques including lateral cerebroventricular injection through a burrhole or cistemal or lumbar puncture or the like (described in Lazorthes et al. Advances in Drug Delivery Systems and Applications in Neurosurgery, 143-192 and Omaya et al., Cancer Drug Delivery, 1: 169-179, the contents of which are incorporated herein by reference). The term “lumbar region” is intended to include the area between the third and fourth lumbar (lower back) vertebrae. The term “cisterna magna” is intended to include the area where the skull ends and the spinal cord begins at the back of the head. The term “cerebral ventricle” is intended to include the cavities in the brain that are continuous with the central canal of the spinal cord. Administration of a compound of Formula (I) to any of the above mentioned sites can be achieved by direct injection of the pharmaceutical composition comprising the compound of Formula (I) or by the use of infusion pumps. For injection, the pharmaceutical compositions of the invention can be formulated in liquid solutions, preferably in physiologically compatible buffers such as Hank's solution or Ringer's solution. In addition, the pharmaceutical compositions may be formulated in solid form and re-dissolved or suspended immediately prior to use. Lyophilized forms are also included. The injection can be, for example, in the form of a bolus injection or continuous infusion (e.g., using infusion pumps) of pharmaceutical composition.

In one embodiment of the invention, the pharmaceutical composition comprising a compound of Formula I is administered by lateral cerebro ventricular injection into the brain of a subject. The injection can be made, for example, through a burr hole made in the subject's skull. In another embodiment, said encapsulated therapeutic agent is administered through a surgically inserted shunt into the cerebral ventricle of a subject. For example, the injection can be made into the lateral ventricles, which are larger, even though injection into the third and fourth smaller ventricles can also be made.

In yet another embodiment, the pharmaceutical composition of the present invention is administered by injection into the cistema magna, or lumbar area of a subject.

For oral administration, the compounds useful in the invention will generally be provided in unit dosage forms of a tablet, pill, dragee, lozenge or capsule; as a powder or granules; or as an aqueous solution, suspension, liquid, gels, syrup, slurry, etc. suitable for ingestion by the patient. Tablets for oral use may include the active ingredients mixed with pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

Pharmaceutical preparations for oral use can be obtained through combination of a compound of Formula (I) with a solid excipient, optionally grinding a resulting mixture, and processing the mixture of granules, after adding suitable additional compounds, if desired, to obtain tablets or dragee cores. Suitable solid excipients in addition to those previously mentioned are carbohydrate or protein fillers that include, but are not limited to, sugars, including lactose, sucrose, mannitol, or sorbitol; starch from corn, wheat, rice, potato, or other plants; cellulose such as methyl cellulose, hydroxypropylmethyl-cellulose or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Capsules for oral use include hard gelatin capsules in which the active ingredient is mixed with a solid diluent, and soft gelatin capsules wherein the active ingredients is mixed with water or an oil such as peanut oil, liquid paraffin or olive oil.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

For transmucosal administration (e.g., buccal, rectal, nasal, ocular, etc.), penetrants appropriate to the barrier to be permeated are used in the formulation. Such penetrants are generally known in the art.

Formulations for rectal administration may be presented as a suppository with a suitable base comprising for example cocoa butter or a salicylate. Formulations suitable for vaginal administration may be presented as pessaries, tampons, creams, gels, pastes, foams or spray formulations containing in addition to the active ingredient such carriers as are known in the art to be appropriate. For intramuscular, intraperitoneal, subcutaneous and intravenous use, the compounds of the invention will generally be provided in sterile aqueous solutions or suspensions, buffered to an appropriate pH and isotonicity. Suitable aqueous vehicles include Ringer's solution and isotonic sodium chloride. Aqueous suspensions according to the invention may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

The suppositories for rectal administration of the drug can be prepared by mixing the drug with a suitable non-irritating excipient which is solid at ordinary temperatures but liquid at the rectal temperatures and will therefore melt in the rectum to release the drug. Such materials are cocoa butter and polyethylene glycols.

The compounds of the inventive method can be delivered transdermally, by a topical route, formulated as applicator sticks, solutions, suspensions, emulsions, gels, creams, ointments, pastes, jellies, paints, powders, or aerosols.

The compounds useful according to the invention may also be presented as aqueous or liposome formulations. Aqueous suspensions of the invention contain a compound of Formula (I) in admixture with excipients suitable for the manufacture of aqueous suspensions. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan monooleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil suspensions can be formulated by suspending a compound of Formula (I) in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

In addition to the formulations described previously, the compounds may also be formulated as a depot preparation. Such long acting formulations may be administered by implantation or transcutaneous delivery (e.g., subcutaneously or intramuscularly), intramuscular injection or a transdermal patch. Thus, for example, the compounds may be formulated with suitable polymeric or hydrophobic materials (e.g., as an emulsion in an acceptable oil) or ion: exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

In general a suitable dose will be in the range of 0.01 to 100 mg per kilogram body weight of the recipient per day, preferably in the range of 0.2 to 10 mg per kilogram body weight per day. The desired dose is preferably presented once daily, but may be dosed as two, three, four, five, six or more sub-doses administered at appropriate intervals throughout the day.

The compounds useful according to the invention can be administered as the sole active agent, or in combination with other known therapeutics to be beneficial in the treatment of neurological disorders. In any event, the administering physician can provide a method of treatment that is prophylactic or therapeutic by adjusting the amount and timing of drug administration on the basis of observations of one or more symptoms (e.g., motor or cognitive function as measured by standard clinical scales or assessments) of the disorder being treated.

Details on techniques for formulation and administration are well described in the scientific and patent literature, see, e.g., the latest edition of Remington's Pharmaceutical Sciences, Maack Publishing Co, Easton Pa. (“Remington's”). Therapeutically effective amounts of a compound of the Formula (I) suitable for practice of the method of the invention may range from about 0.5 to about 25 milligrams per kilogram (mg/kg). A person of ordinary skill in the art will be able without undue experimentation, having regard to that skill and this disclosure, to determine a-therapeutically effective amount of a particular compound of the Formula (I) for practice of this invention.

After a pharmaceutical composition of the inventive method has been formulated in an acceptable carrier, it can be placed in an appropriate container and labeled for treatment of an indicated condition. For administration of the compounds of the Formula (I), such labeling would include, e.g., instructions concerning the amount, frequency and method of administration. In one embodiment, the invention provides for a kit for inhibiting or reversing AP-induced weight gain in a human which includes a compound of the Formula (I) and instructional material teaching the indications, dosage and schedule of administration of the compound of the Formula (I).

In one embodiment, the invention provides methods as described herein, further comprising identifying a subject in need of prevention or treatment of a neurological condition. In another embodiment, the invention provides a method as described above, further comprising the step of obtaining the compound of Formula (I). In one embodiment of the methods described herein, the subject is a mammal. In a further embodiment, the subject is a human

V. Compound Toxicity

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. See, e.g. Fingl el al., In. The Pharmacological Basis of Therapeutics, Ch.1, p. 1, 1975. The exact formulation, route of administration and dosage can be chosen by the individual physician in view of the patient's condition and the particular method in which the compound is used.

VI. Experiment Details

A library of compounds was screened to identify compounds that reduce the polyglutamine-induced neurodegeneration and behavior in a Drosophila HD model. The Drosophila HD model used to screen compounds can be generated using standard genetic engineering technology, such as those described below.

A “tissue-specific” expression control sequence, as used herein, refers to expression control sequences that drive expression in one tissue or a subset of tissues, while being essentially inactive in at least one other tissue. “Essentially inactive” means that the expression of a sequence operatively linked to a tissue-specific expression control sequence is less than 5% of the level of expression of that sequence in that tissue where the expression control sequence is most active. Preferably, the level of expression in the tissue is less than 1% of the maximal activity, or there is no detectable expression of the sequence in the tissue. “Tissue-specific expression control sequences” include those that are specific for organs such as the eye, wing, notum, brain, as well as tissues of the central and peripheral nervous systems. Tissue-specific expression control sequences of the invention either can be used as a “driver” in the Gal4-UAS system, or alternatively can be inserted upstream from a transgene to control its expression in a cis acting manner.

Examples of tissue specific control sequences include but are not limited to: promoters/enhancers important in eye development, such as sevenless (Bowtell et al., Genes Dev. 2:620-34 (1988)), eyeless (Bowtell et al., Proc. Natl. Acad. Sci. U.S.A. 88:6853-7 (1991)), and GMR/glass (Quiring et al., Science 265:785-9 (1994)); promoters/enhancers derived from any of the rhodopsin genes, that are useful for expression in the eye; enhancers/promoters derived from the dpp or vestigial genes useful for expression in the wing (Staehling-Hampton et al., Cell Growth Differ. 5:585-93 (1994), Kim et al., Nature 382:133-8 (1996)); promoters/enhancers specific for nerve, e.g., elav (Yao and White, J. Neurochem. 63:41-51 (1994)) which is specific for pan-neuronal expression in post-mitotic neurons, scabrous (sca) (Song et al., Genetics 162:1703-24 (2002) which is specific for pan-neuronal expression in neuroblasts to neurons, APPL (Martin-Morris and White, Development 110: 185-95 (1990)), Nervana 2 (Nrv2)(Sun et al., Proc. Nat'l. Acad. Sci. U.S.A. 96:10438-43 (1999)) which is specific for expression in the central nervous system, Cha (Barber et al., J. Comp. Neurol. 22:533-43 (1989)) which is specific for cholinergic neurons, TH (Friggi-Grelin et al., J. Neurobiol. 54:618-27 (2003)) which is specific for dopaminergic neurons, CaMKII (Takmatsu et al., Cell Tissue Res. 310:237-52 (2002)) which is specific for central nervous system of embryos and larvae as well as brain, throacic ganglion and gut of adult, P (Gendre et al., Development 131:83-92 (2004)) which is specific for pharangeal sensory neurons, Dmef2 (Mao et al., Proc. Natl. Acad. Sci. USA 101:198-203 (2004), Gal4 line named “P247”) which is specific for adult mushroom bodies of the brain, and promoters/enhancers derived from other neural-specific genes; and gcm (Dumstrei et al., J. Neurosci. 23:3325-35 (2003)) which is specific for glial cells; all of which references are incorporated by reference herein. Other examples of expression control sequences include, but are not limited to the heat shock promoters/enhancers from the hsp70 and hsp83 genes, useful for temperature induced expression; and promoters/enhancers derived from ubiquitously expressed genes, such as tubulin, actin, or ubiquitin.

The present invention utilizes transgenic flies that have incorporated into their genome a DNA sequence that encodes exons 1-4 of huntingtin.

The transgenic flies for screening compounds for treating neurological diseases can be generated by any means known to those skilled in the art. Methods for production and analysis of transgenic Drosophila strains are well established and described in Brand et al., Methods in Cell Biology 44:635-654 (1994); Hay et al., Proc. Natl. Acad. Sci. USA 94(10):5195-200 (1997); and in Drosophila: A Practical Approach (ed. D. B. Roberts), pp 175-197, IRL Press, Oxford, UK (1986), herein incorporated by reference in their entireties.

In general, to generate a transgenic fly, a transgene of interest is stably incorporated into a fly genome. Any fly can be used, however a preferred fly of the present invention is a member of the Drosophilidae family. An exemplary fly is Drosophila melanogaster.

A variety of transformation vectors are useful for the generation of transgenic flies, and include, but are not limited to, vectors that contain transposon sequences, which mediate random integration of a transgene into the genome, as well as vectors that use homologous recombination (Rong and Golic, Science 288:2013-2018 (2000)). A preferred vector of the present invention is pUAST (Brand and Perrimon, Development 118:401-415 (1993)) which contains sequences from the transposable P-element which mediate insertion of a transgene of interest into the fly genome. Another preferred vector is PdL, which is able to yield doxycycline-dependent overexpression (Nandis, Bhole and Tower, Genome Biology 4 (R8):1-14, (2003)). Yet another preferred vector is pExP-UAS because of its ease of cloning and mapping genomic location.

P-element transposon mediated transformation is a commonly used technology for the generation of transgenic flies and is described in detail in Spradling, P element mediated transformation, in Drosophila: A Practical Approach (ed. D. B. Roberts), pp 175-197, IRL Press, Oxford, UK (1986), herein incorporated by reference. Other transformation vectors based on transposable elements include, for example, the hobo element (Blackman et al., Embo J. 8:211-7 (1989)), the mariner element (Lidholm et al., Genetics 134:859-68 (1993)), the hermes element (O'Brochta et al., Genetics 142:907-14 (1996)), the Minos element (Loukeris et al., Proc. Natl. Acad. Sci. USA 92:9485-9 (1995)), or the PiggyBac element (Handler et al., Proc. Natl. Acad. Sci. USA 95:7520-5 (1998)). In general, the terminal repeat sequences of the transposon that are required for transposition are incorporated into a transformation vector and arranged such that the terminal repeat sequences flank the transgene of interest. It is preferred that the transformation vector contains a marker gene used to identify transgenic animals. Commonly used, marker genes affect the eye color of Drosophila, such as derivatives of the Drosophila white gene (Pirrotta V., & C. Brockl, EMBO J. 3(3):563-8 (1984)) or the Drosophila rosy gene (Doyle W. et al., Eur. J Biochem. 239(3):782-95 (1996)) genes. Any gene that results in a reliable and easily measured phenotypic change in transgenic animals can be used as a marker. Examples of other marker genes used for transformation include the yellow gene (Wittkopp P. et al., Curr Biol. 12(18):1547-56 (2002)) that alters bristle and cuticle pigmentation; the forked gene (McLachlan A., Mol Cell Biol. 6(1):1-6 (1986)) that alters bristle morphology; the Adh+ gene used as a selectable marker for the transformation of Adh− strains (McNabb S. et al., Genetics 143(2):897-911 (1996)); the Ddc+ gene used to transform Ddc^{ts2} mutant strains (Scholnick S. et al., Cell 34(1):37-45(1983)); the lacZ gene of E. coli; the neomycin^{R} gene from the E.coli transposon Tn5; and the green fluorescent protein (GFP; Handler and Harrell, Insect Molecular Biology 8:449-457 (1999)), which can be under the control of different promoter/enhancer elements, e.g. eyes, antenna, wing and leg specific promoter/enhancers, or the poly-ubiquitin promoter/enhancer elements.

Plasmid constructs for introduction of the desired transgene are coinjected into Drosophila embryos having an appropriate genetic background, along with a helper plasmid that expresses the specific transposase needed to incorporate the transgene into the genomic DNA. Animals arising from the injected embryos (G0 adults) are selected, or screened manually, for transgenic mosaic animals based on expression of the marker gene phenotype and are subsequently crossed to generate fully transgenic animals (G1 and subsequent generations) that will stably carry one or more copies of the transgene of interest.

Binary systems are commonly used for the generation of transgenic flies, such as the UAS/GAL4 system. This is a well established system which employs the UAS upstream regulatory sequence for control of promoters by the yeast GAL4 transcriptional activator protein, as described in Brand and Perrimon, Development 118:401-15 (1993)) and Rorth et al, Development 125:1049-1057 (1998), herein incorporated by reference in their entireties. In this approach, transgenic Drosophila, termed “target” lines, are generated where the gene of interest (e.g., huntington) is operatively linked to an appropriate promoter (e.g., hsp70TATA box, see Brand and Perrimon, Development 118:401-15 (1993)) controlled by UAS. Other transgenic Drosophila strains, termed “driver” lines, are generated where the GAL4 coding region is operatively linked to promoters/enhancers that direct the expression of the GAL4 activator protein in specific tissues, such as the eye, antenna, wing, or nervous system. The gene of interest is not expressed in the “target”0 lines for lack of a transcriptional activator to “drive” transcription from the promoter joined to the gene of interest. However, when the UAS-target line is crossed with a GAL4 driver line, the gene of interest is induced. The resultant progeny display a specific pattern of expression that is characteristic for the GAL4 line.

The technical simplicity of this approach makes it possible to sample the effects of directed expression of the gene of interest in a wide variety of tissues by generating one transgenic target line with the gene of interest, and crossing that target line with a panel of pre-existing driver lines. Numerous GAL4 driver Drosophila strains with specific drivers have been described in the literature and others can readily be prepared using established techniques (Brand and Perrimon, Development 118:401-15 (1993)). Driver strains for use with the invention include, for example, apterous-Gal4 for expression in wings, brain, and intemeurons; elav-Gal4 for pan-neuronal expression in post-mitotic neurons; scabrous-Gal4 for pan-neuronal expression in the developing nervous system from neuroblasts to neurons; sevenless-Gal4, eyeless-Gal4, and GMR-Gal4 for expression in eyes; Nervana 2-Gal4 for expression in the central nervous system; Cha—(choline acetyltransferase) Gal4 for expression in cholinergic neurons, TH—(tyrosine hydroxylase) for expression in dopaminergic neurons; CaMKII—(calmodulin dependent kinase II) for expression in the central nervous system of embryos and larvae as well as the brain, throacic ganglion, and gut of adults; P-Gal4 for expression in pharangeal sensory neurons; and gcm-Gal4 for expression in glial cells.

In a preferred embodiment, transgenic Drosophila are produced using the UAS/GAL4 control system. Briefly, to generate a transgenic fly that expresses a mutant form of huntingtin containing a 128Q repeat in exon 1, a DNA sequence encoding htt128Q is cloned into a vector such that the sequence is operatively linked to the GAL4 responsive element UAS. Vectors containing UAS elements are readily available in the art, such as the pUAST vector (Brand and Perrimon, Development 118:401-415 (1993)), which places the UAS sequence element upstream of the transcribed region. The DNA is cloned using standard methods (Sambrook et al., Molecular Biology: A laboratory Approach, Cold Spring Harbor, N.Y. (1989); Ausubel, et al., Current protocols in Molecular Biology, Greene Publishing, Y, (1995)) and is described in more detail under the Molecular Techniques section of the present application. After cloning the DNA into appropriate vector, such as pUAST, the vector is injected into Drosophila embryos (e.g. yw embryos) by standard procedures (Brand et al., Methods in Cell Biology 44:635-654 (1994); Hay et al., Proc. Natl. Acad. Sci. USA 94:5195-200 (1997)) to generate transgenic Drosophila.

When the binary UAS/GAL4 system is used, the transgenic progeny can be crossed with Drosophila driver strains to assess the presence of an altered phenotype. A preferred Drosophila comprises the eye specific driver strain GMR-GAL4, which enables identification and classification of transgenics flies based on the severity of a eye phenotype.

To evaluate locomotor and behavioral phenotypes (e.g., climbing assay), an elav-Gal4 driver strain is used in the cross. Ectopic overexpression of htt128Q in Drosophila central nervous system (CNS) results in locomotor deficiencies, such as impaired movement, climbing and flying.

Expression of proteins such as huntingtin in transgenic flies is confirmed by standard techniques, such as Western blot analysis or by immunostaining of fly tissue cross-sections, both of which are described below.

Locomotor Phenotypes

Locomotor phenotypes can be assessed, for example, as described in U.S. Application Nos. 2004/0076583, 2004/0076318, and 2004/0076999, each of which is hereby incoporated by reference in its entirety. For example, locomotor ability can be assessed in a climbing assay by placing flies in a vial, knocking them to the bottom of the vial, then counting the number of flies that climb past a given mark on the vial during a defined period of time. In this example, 100% locomotor activity of control flies is represented by the number of flies that climb past the given mark, while flies with an altered locomotor activity can have 80%, 70%, 60%, 50%, preferably less than 50%, or more preferably less than 30% of the activity observed in a control fly population.

In one aspect, the traits are measured by detecting and serially analyzing the movement of a population of flies in containers, e.g., vials. Movement of the flies can be monitored by a recording instrument, such as a CCD-video camera, the resultant images can be digitized, analyzed using processor-assisted algorithms as described herein, and the analysis data stored in a computer-accessible manner. For example, in measuring traits related to fly movement, the trajectory of each animal may be monitored by calculation of one or more variables (e.g., speed, vertical only speed, vertical distance, turning frequency, frequency of small movements, etc.) for the animal. Values of such a variable are then averaged for population of animals in the vial and a global value is obtained describing the trait for each population (e.g., parental stock flies and transgenic flies).

“Movement trait data” as used herein refers to the measurements made of one or more movement traits. Examples of “movement trait data” measurements include, but are not limited to X-pos, X-speed, speed, turning, stumbling, size, T-count, P-count, T-length, Cross150, Cross250, and F-count. Descriptions of these particular measurements are provided below.

Examples of such “movement traits” include, but are not limited to:

a) total distance (average total distance traveled over a defined period of time);

b) X only distance (average distance traveled in X direction over a defined period of time;

c) Y only distance (average distance traveled in Y direction over a defined period of time);

d) average speed (average total distance moved per time unit);

e) average X-only speed (distance moved in X direction per time unit);

f) average Y-only speed (distance moved in Y direction per time unit);

g) acceleration (the rate of change of velocity with respect to time);

h) turning;

i) stumbling;

j) spatial position of one fly to a particular defined area or point (examples of spatial position traits include (1) average time spent within a zone of interest (e.g., time spent in bottom, center, or top of a container; number of visits to a defined zone within container); (2) average distance between a fly and a point of interest (e.g., the center of a zone); (3) average length of the vector connecting two sample points (e.g., the line distance between two flies or between a fly and a defined point or object); (4) average time the length of the vector connecting the two sample points is less than, greater than, or equal to a user define parameter; and the like);

k) path shape of the moving fly, i.e., a geometrical shape of the path traveled by the fly (examples of path shape traits include the following: (1) angular velocity (average speed of change in direction of movement); (2) turning (angle between the movement vectors of two consecutive sample intervals); (3) frequency of turning (average amount of turning per unit of time); (4) stumbling or meandering (change in direction of movement relative to the distance); and the like. This is different from stumbling as defined above. Turning parameters may include smooth movements in turning (as defined by small degrees rotated) and/or rough movements in turning (as defined by large degrees rotated).

Movement traits can be quantified, for example, using the following parameters:

X-Pos: The X-Pos score is calculated by concatenating the lists of x-positions for all trajectories and then computing the average of all values in the concatenated list.

X-Speed: The X-Speed score is calculated by first computing the lengths of the x-components of the speed vectors by taking the absolute difference in x-positions for subsequent frames. The resulting lists of x-speeds for all trajectories are then concatenated and the average x-speed for the concatenated list is computed.

Speed: The Speed score is calculated in the same way as the X-Speed score, but instead of only using the length of the x-component of the speed vector, the length of the whole vector is used. That is, [length]=square root of ([x-length]²+[y-length]²).

Turning: The Turning score is calculated in the same way as the Speed score, but instead of using the length of the speed vector, the absolute angle between the current speed vector and the previous one is used, giving a value between 0 and 90 degrees.

Stumbling: The Stumbling score is calculated in the same way as the Speed score, but instead of using the length of the speed vector, the absolute angle between the current speed vector and the direction of body orientation is used, giving a value between 0 and 90 degrees.

Size: The Size score is calculated in the same way as the Speed score, but instead of using the length of the speed vector, the size of the detected fly is used.

T-Count: The T-Count score is the number of trajectories detected in the movie.

P-Count: The P-Count score is the total number of points in the movie (i.e., the number of points in each trajectory, summed over all trajectories in the movie).

T-Length: The T-Length score is the sum of the lengths of all speed vectors in the movie, giving the total length all flies in the movie have walked.

Cross150: The Cross150 score is the number of trajectories that either crossed the line at x=150 in the negative x-direction (from bottom to top of the vial) during the movie, or that were already above that line at the start of the movie. The latter criterion was included to compensate for the fact that flies sometimes don't fall to the bottom of the tube. In other words this score measures the number of detected flies that either managed to hold on to the tube or that managed to climb above the x=150 line within the length of the movie.

Cross250: The Cross250 score is equivalent to the Cross150 score, but uses a line at x=250 instead.

F-Count: The F-Count score counts the number of detected flies in each individual frame, and then takes the maximum of these values over all frames. It thereby measures the maximum number of flies that were simultaneously visible in any single frame during the movie.

The assignment of directions in the X-Y coordinate system is arbitrary. For purposes of this disclosure, “X” refers to the vertical direction (typically along the long axis of the container in which the flies are kept) and “Y” refers to movement in the horizontal direction (e.g., along the surface of the vial).

Behavioral Phenotypes

Neuronal degeneration in the central nervous system will give rise to behavioral deficits, including but not limited to locomotor deficits, that can be assayed and quantitated in both larvae and adult Drosophila. For example, failure of Drosophila adult animals to climb in a standard climbing assay (see, e.g. Ganetzky and Flannagan, J. Exp. Gerontology 13:189-196 (1978);. LeBourg and Lints, J. Gerontology 28:59-64 (1992)) is quantifiable, and indicative of the degree to which the animals have a motor deficit and neurodegeneration. Neurodegenerative phenotypes include, but are not limited to, progressive loss of neuromuscular control, e.g. of the wings; progressive degeneration of general coordination; progressive degeneration of locomotion, and progressive loss of appetite. Other aspects of fly behavior that can be assayed, include but are not limited to circadian behavioral rhythms, feeding behaviors, inhabituation to external stimuli, and odorant conditioning. All of these phenotypes are measured by one skilled in the art by standard visual observation of the fly.

Another neural degeneration phenotype, is a reduced life span, for example, the Drosophila life span can be reduced by 10-80%, e.g., approximately, 30%, 40%, 50%, 60%, or 70%.

Memory and Learning Phenotypes

In Drosophila, the best characterized assay for associative learning and memory is an odor-avoidance behavioral task (T. Tully, et al. J. Comp. Physiol. A157, 263-277 (1985), incorporated herein by reference). This classical (Pavlovian) conditioning involves exposing the flies to two odors (the conditioned stimuli, or CS), one at a time, in succession. During one of these odor exposures (the CS+), the flies are simultaneously subjected to electric shock (the unconditioned stimulus, or US), whereas exposure to the other odor (the CS−) lacks this negative reinforcement. Following training, the flies are then placed at a “choice point,” where the odors come from opposite directions, and expected to decide which odor to avoid. By convention, learning is defined as the fly's performance when testing occurs immediately after training. A single training trial produces strong learning: a typical response is that >90% of the flies avoid the CS+. Performance of wild-type flies from this single-cycle training decays over a roughly 24-hour period until flies once again distribute evenly between the two odors. Flies can also form long-lasting associative olfactory memories, but normally this requires repetitive training regimens.

A common feature of many neurodegenerative diseases is the presence of protein aggregation in the brain. Examples of neurodegenerative diseases characterized by protein aggregation include Alzheimer's disease, Parkinson's disease, amyotrophic lateral sclerosis, Pick's disease, prion diseases, and other spongiform encephalopathies.

The invention is illustrated by the following examples which are not intended to be limiting in any way.

### EXAMPLES

### Example 1

### Synthesis of Triarylmethane Compounds

Abbreviations

Abbreviations used in the following Examples include:
AcCl Acetyl chloride
Bn Benzyl
Celite™ Diamotaceous earth
CH₂Cl₂ Dichloromethane (Methylene Chloride)
CHCl₃ Chloroform
CI (c.i.) Chemical Ionization
DCC N-cyclohexyl-N′-(4-diethylaminocyclohexyl)-carbodiimide
1,2 DCE 1,2-Dichloroethane
d Doublet
dd Double Doublet
DIEA Di-isopropylethyl amine
DMAP 4-Dimethylamino Pyridine
DME 1,2 Dimethoxyethane
DMF Dimethylformamide
DMSO Dimethyl sulfoxide
EDC 1-(3-Dimethylaminopropyl)-3-ethylcarbodiimide Hydrochloride
EtOAc Ethyl Acetate
EtOH Ethyl Alcohol or Ethanol
Et₂O Ethyl Ether
Et₃N Triethylamine
EtNⁱPr₂ N,N Di-isopropylethylamine (Hünigs' Base)
g gram(s)
HCl Hydrochloric acid
HNO₃ Nitric acid
HN(Me)OMe N,O-Dimethylhydroxylamine
H₂SO₄ Sulfuric acid
HOBt 1-Hydroxybenzotriazole
HPLC High Pressure Liquid Chromatography
h Hour(s)
hr Hour(s)
K₂CO₃ Potassium Carbonate
m Multiplet
MeOH Methyl Alcohol (Methanol)
MeCN Acetonitrile
min Minute(s)
mmol millimoles
mmole millimoles
MS Mass Spectrometry
MsOH Methane sulfonic acid
MTBE Methyl tert-butyl ether
NMR Nuclear Magnetic Resonance
NMO N-Methyl Morpholine
o/n overnight
ⁱPrOH Iso-propanol
PPAA 1-Propanephosphonic Acid Cyclic Anhydride
PyBOP® Benzotriazol-1-yl-oxytripyrrolidinophosphonium hexafluorophosphate
PyBroP® bromotripyrrolidino phosphonium hexafluorophosphate
q Quartet
RT (or rt) room temperature (about 20-25° C.)
s Singlet
sat. Saturated
t Triplet
TBAF Tetra-butyl Ammonium Fluoride
TFA Trifluoroacetic Acid
THF Tetrahydrofuran
p-TsOH para-Toluene Sulphonic Acid (p-Tosic Acid)
v/v volume/volume
wt/v weight/volume
   (a) Preparation of Triarylmethanols of Formula (IX) (General Method 1)

A solution of the respective Grignard reagent (1.3 eq.) in THF or diethyl ether was added dropwise with stirring to an appropriately substituted benzophenone (1.0 eq.) in MTBE at room temperature. The reaction mixture was stirred overnight and quenched with 2 N aqueous HCl at 0° C. The organic phase was separated and the aqueous phase was extracted with ethyl acetate or MTBE. The combined organic phases were washed with water and brine, and dried over MgSO₄. Evaporation of the solvent gave the respective crude material, which usually was purified by column chromatography (silica gel, EtOAc:heptane, 1:3-1:1).

(b) Preparation of Triarylchloromethanes of Formula (X) (General Method 2)

To a stirred solution of the respective triarylmethanol in methylene chloride at room temperature was added dropwise an excess (1.5-2.5 times) of acetyl chloride. The reaction mixture was stirred at room temperature overnight. The excess of acetyl chloride and the solvent were evaporated in vacuum; residual solvents were co-evaporated with toluene to remove acetic acid. If a solid residue was formed, it was washed with heptanes. To avoid hydrolysis of these sensitive triarylchloromethanes, they were used for further reactions after being characterized by NMR without purification.

(c) Preparation of Triarylmethylamines of Formula (I) (General Method 3)

To a solution of the appropriate triarylmethyl chloride (1 eq.) in anhydrous acetonitrile was added the desired amine/heterocycle (1.3 eq.) and triethylamine (5 eq.). The reaction mixture was stirred at 50-80° C. for 4 h or at room temperature overnight. The solvent was evaporated and the residue was dissolved in EtOAc. The mixture was washed two times with water and then with brine, dried over MgSO₄, and concentrated in vacuum. The residue was purified by column chromatography (silica gel, EtOAc:heptane, 1:3-1:1) or, if the residue was solid, recrystallized from heptane:EtOAc (1:3).

(d) (2-Chlorophenyl)-diphenylmethanol from Clotrimazole.

Clotrimazole (10.0 g, 30 mmol) was refluxed in 1 M aqueous HCl for 3 h. The reaction mixture was cooled to room temperature and extracted with EtOAc (3×50 mL). The organic solution was dried over MgSO₄, the solvent was evaporated in vacuum, and the residue was recrystallized from EtOAc:heptane to give (2-Chlorophenyl)-diphenylmethanol as white crystals, yield: 7.23 g (85%), m.p. 87° C.; ¹H NMR (300 MHz, CDCl₃/TMS): δ 4.50 (br s, 1H), 6.82 (d, J=7.5 Hz, 1H), 7.17 (t, J=7.4 Hz, 1H), 7.29-7.49 (m, 12H); ¹³C NMR (75 MHz, CDCl₃/TMS): δ 82.6, 126.3, 127.3, 127.7, 127.9, 129.0, 131.2, 131.4, 133.1, 143.6, 145.4; MS (HR, ci, pos): M⁺ calcd for C₁₉H₁₅OCl: 294,0811. found: 294.0817; chromatography (HPLC), purity: 99.6%.

Exemplary compounds prepared according to General Method 3 include the following:

(e) 1-[(3-Trifluoromethylphenyl)-diphenylmethyl]-1H-imidazole.

White solid; 1.8 g, yield: 47%; m.p. 157° C.; ¹H NMR (300 MHz, CDCl₃/TMS): δ 6.71-7.65 (m, 17H); ¹³C NMR (75 MHz, CDCl₃/TMS): δ 74.8, 121.3, 124 (q), 124.8 (m), 125.7 (m), 128.1, 128.2, 128.4, 128.6, 129.4, 130.30 (q, J=30 Hz), 133.0, 138.6, 141.4, 143.5; MS (HR, ci, pos): [M+H]⁺ calcd for C₂₃H₁₈F₃N₂: 379.1422. found: 379.1426; chromatography (HPLC), purity: 98.7%.

(f) 4-[(2-Chlorophenyl)-diphenylmethyl]-morpholine

White solid; yield: 0.800 g, 69%; m.p. 205° C.; ¹H NMR (300 MHz, CDCl₃/TMS): δ 1.68 (br, 2H), 2.70 (br, 2H), 3.75 (m, 4H), 7.10-7.47 (m, 14); ¹³C NMR (75 MHz, CDCl₃/TMS): δ 48.5, 67.5, 75.5, 126.6, 127.0, 127.5 (br), 128.0, 130.0 (br), 132.5, 134.3, 136.8, 141.8; MS (HR, ci, pos): M⁺ calcd for C₂₃H₂₂OCl N: 363.1390. found: 363.1394; chromatography (HPLC), purity: 99.3%.

(g) 4-[(3-Trifluoromethylphenyl)-diphenylmethyl]-morpholine

White solid; 0.600 g, yield: 52%; m.p. 158-160° C.; ¹H NMR (300 MHz, CDCl₃/TMS): δ 1.40-3.20 (br s, 4H), 3.88 (s, 4H), 7.23-7.47 (m, 12H), 7.71 (s, 1H), 7.85 (s, 1H), 7.85 (s, 1H), ¹³C NMR (75 MHz, CDCl₁₃/TMS): δ 48.5, 67.5, 76.9, 122.9 (d), 125.6 125.7 (t), 126.4, 127.7, 128.0, 129.2, 130.1, 132.6; ¹⁹F NMR (282 MHz, CDCl₃): δ −62.9; MS (HR, ci, pos): M⁺ calcd for C₂₄H₂₂ONF₃: 397.1653. found: 397.1660; chromatography (HPLC), purity: 98.9%.

(h) 1-[Bis-(4-fluorophenyl)-phenylmethyl]1H-imidazole

White solid; 1.2 g, yield: 55%; m.p. 135° C.; ¹H NMR (300 MHz, CDCl₃/TMS): δ 6.82 (s, 1H), 7.03-7.13 (m, 11H), 7.37-7.46 (m, 4H); ¹³C NMR (75 MHz, CDCl₃/TMS): δ 74.3, 115.0 (d, J=22 Hz), 121.4, 128.2, 128.3, 128.7, 129.5, 131.4 (d, J=8 Hz), 138.2 (d, 3 Hz), 138.7, 142.1, 162.1 (d, J=249 Hz); MS (HR, ci, pos): [M+H]⁺ calcd for C₂₂H₁₇N₂F₂: 347.1360. found: 347.1343; chromatography (HPLC), purity: 98.6%.

(i) 1-[Bis-(4-fluorophenyl)-(3-trifluoromethyl-phenyl)-methyl]1H-imidazole

### Example 2

### Production of Transgenic Drosophila Model of Huntington's Disease

Female “activator” or “driver” flies were engineered to contain the neuronal specific drivers elav-GAL4 or nirvana-GAL4 in a heat shock-hid Y (phs-hidY or HS-hid+ on Y chomosome) background (Starz-Gaiano et al., 2001). The hid gene is a pro-apoptotic gene, which is expressed in this line by a heat inducible promoter. These flies can be heat shocked in bottles twice over 2 days for 2 hours to kill male larvae and thereby produce massive numbers of elav-GAL4 or nirvana-GAL4 virgin females.

For the mass production of males (either UAS-htt128Q[F27B]/UAS-htt128Q[F27B], the disease transgenes were moved into lines that contain an attached X-chromosome with the same phs-hid element (X^{XPhs-hidY}) used above. In this scheme the hid gene is present only in female flies and therefore is used to produce large numbers of male flies by heat shock. The massive collection of virgin female elav-GAL4 flies are crossed with the massive collection of males that contain the silent disease-transgene. Their progeny, the F1 generation, are the “assay flies” (e.g., elav-GAL4UAS-htt128Q[F27B]). These assay flies are robotically distributed into drugged assay vials.

The HD Drosophila model express exons 1 to 4 of the human huntingtin gene with 128Q repeats in exon 1. The construct was cloned into the Drosophila transformation vector, pUAST. These were then crossed to GAL4 driver lines that direct expression either to the eye (GMR-GAL4 promoter) or to neurons (elav-GAL4 promoter). The model shows degeneration of photoreceptors when the transgene was expressed in the eye. Progressive degeneration of the photoreceptors was observed after several days of adult life. The properties of the HD128Q flies were consistent with those reported by others for two other strains in which exon-1 containing expanded repeats was expressed in the eye (Jackson et al., 1998; Steffan et al., 2001—US20040142859). To test the effects on motor function of the HD mutation, HD128Q lines were examined in which the transgene was expressed in all neurons. Motor function was assayed by tapping flies in a vial to the bottom and measuring the number of flies that are able to climb to a specified height within a specific length of time. When tapped to the bottom of a vial, wild-type flies rapidly climb to the top, where most of them remain. When assayed by this manual procedure, the HD128Q transgenic lines showed normal behavior early in life. Starting at 10-12 days of life, however, the transgenic flies were observed to make short abortive climbs and fall back to the bottom. The decline in motor performance was progressive, leading to early death. Examination of the HD128Q flies under the microscope revealed that their motor dysfunction was the result of spontaneous and uncoordinated movement of the limbs, which inhibited normal locomotion. Examination of the neurons in these flies showed progressive degeneration and loss of particular neurons, accompanied by the formation of nuclear inclusions that stain positively for molecular chaperones, ubiquitin, and components of the proteasome. The HD128Q model thus has a functional deficit that in its phenotypic and neuropathological characteristics appears to be related to that seen in the human disease. Moreover, as the deficit is quantifiable and reproducible, it is suitable for automated high-throughput screening.

### Example 3

### Assay of Locomotor Activity Using the Transgenic Drosophila HD Model

Standard environmental conditions were followed to establish that different metrics detect differences between disease and control flies. A comparison of compound-treated HD flies and untreated HD flies was also included. The positive control compound tested was the Histone Deacetylase (HDAC) inhibitor Trichostatin-A (TSA).

In an assay run (typically corresponding to one day of an entire multi-day assay) the following process was followed. Each tube was dropped onto a surface, causing the flies to fall to the bottom of the tube and be subjected to impact shock stimulation. Their immediate negative geotaxis response to that event was thereafter captured in a 7.5 second movie. For each tube, this step was repeated five times, giving a total of five repeat videos per tube and run. The raw data for each video were analyzed and trajectories following the movements of individual flies in a tube were generated. The method for analyzing fly trajectories is described in published U.S. Patent Application 2004/0076318A1, which is hereby incorporated by reference in its entirety. The trajectories consisted of a list of x- and y-coordinates mapping out the positions of all the flies in the video, as well as measurements of size, orientation, length and width of the flies, with one list entry for every frame from when the fly started moving in one frame until it stopped in another. These data were then used to provide different scoring metrics for the test flies' behavior. The definitions of the 11 metrics developed are summarized above.

The movies were first scored individually to give one value per metric and movie. Average values for all metrics were then calculated from the movie score values over all five repeat videos for a vial, resulting in one value per tube and metric. In a typical assay run, there were several tubes subjected to the same treatment. The data values from replicate tubes were subsequently used to calculate treatment means and standard errors for each specific treatment.

Average speed of the Drosophila flies were measured by a robot at both early and late speed, and early and late speed summary metrics were created. Early speed was the average speed of eight replicate vials, ten flies/vial, four repeat videos per trial day, averaged over days 1-7. Late speed was the same but measured and averaged over days 8-10. Effect sizes were the differences between average mean speeds of the summary metrics (i.e., differences between DMSO-carrier control and test drug) divided by the pooled standard deviation in the whole assay.

TABLE 1

| | | Early Speed | | Late Speed | |
|---|---|---|---|---|---|
| | | Effect | | Effect | |
| Dose | Campaign | Size | Percent TSA | Size | Percent TSA |
| 50 | C00083 | 0.73 | 26.3 | 1.46 | 38.8 |
| 50 | C00084 | 0.88 | 25.4 | 1.39 | 31.5 |
| 50 | Summary | 0.8 | 25.9 | 1.43 | 35.7 |
| 100 | C00074 | 1.43 | 54.7 | 1.32 | 40.9 |
| 100 | C00076 | 0.3 | 15.5 | 0.39 | 22.3 |
| 100 | C00083 | 0.96 | 34.9 | 0.85 | 22.6 |
| 100 | C00084 | 1.92 | 55.2 | 3.68 | 83.5 |
| 100 | Summary | 1.13 | 39.6 | 1.42 | 39.6 |
| 150 | C00083 | 0.63 | 23 | 0.82 | 21.8 |
| 150 | C00084 | 2.3 | 66.5 | 3.36 | 76.2 |
| 150 | Summary | 1.35 | 41.6 | 1.8 | 42.7 |

In Table 1 the “summary” is the average of the effect sizes from multiple different campaigns (e.g., C00083) in which clotrimazole was tested. Each individual campaign measured 1536 vials over 10 days, one run per day. “Percent TSA” provides a reference as to how the compounds performed in a particular campaign compared to the positive control, which is TSA (trichostatin-A, an HDAC inhibitor); thus “percent TSA” in Table 1 is essentially the effect size as a percentage of the positive control. “Dose” is the final concentration of the drug or control compound in the food. The phenotype measured is the progressive decline in motor coordination and reduction in average walking speed following stimulation (tapping of the vial). The flies also have a reduced lifespan. The calculated summary effect size(s) and percent TSA(s) for multiple campaigns are a weighted average across experiments, using sample size as weight. Not all campaigns have the same number of replicas. The more replicas then a greater weight is attributed.
Table 1 lists the results of a climbing assay using clotrimazole, showing a dose-dependent effect.

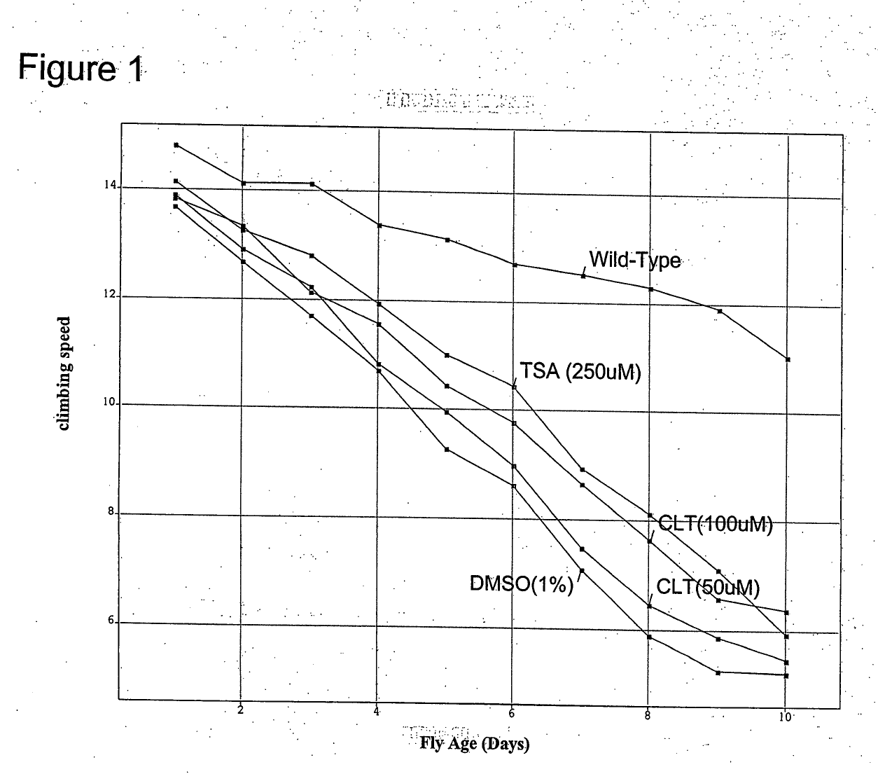

FIG. 1 depicts the decline in speed vs. carrier control HD flies.

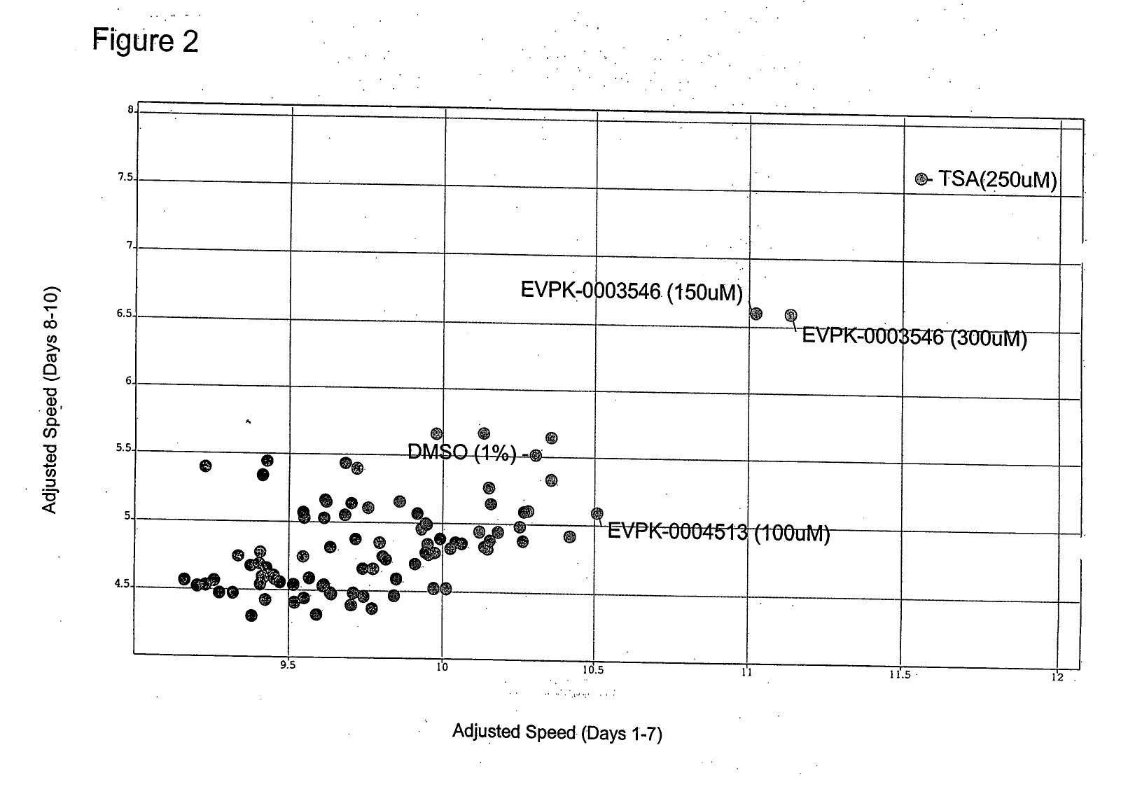

FIG. 2 is a scatter plot depicting early speed (1-7) vs. late speed (8-10 days) for several pounds of Formula (I). Table 2 depicts the effect size (ES), percent TSA for the early and speeds for campaigns of Drosophila using 1-(triphenylmethyl)imidazole as the active agent.

TABLE 2

| | | Early Speed | | | Late Speed | | |
|---|---|---|---|---|---|---|---|
| | | Adj. | | Percent | Adj. | | Percent |
| Conc. | Campaign ID | Value | ES | TSA | Value | ES | TSA |
| 100 | C00080 | 10.62 | 0.77 | 31.0 | 5.81 | 0.86 | 37.3 |
| 100 | C00084 | 10.71 | 0.33 | 9.6 | 5.41 | 0.62 | 14.1 |

Table 3 depicts the effect size (ES) and percent TSA for the early and late speeds for paigns of Drosophila using 1-(3-(trifluoromethyl)trityl)imidazole as the active agent.

TABLE 3

| Conc. | Campaign ID | ES | Percent TSA | ES | Percent TSA |
|---|---|---|---|---|---|
| 300 | C00080 | 1.76 | 71% | 2.88 | 125% |
| 300 | C00084 | 0.73 | 21% | 1.62 | 37% |
| 300 | C00085 | 1.36 | 71% | 1.96 | 94% |

Table 4 depicts the dose (food concentration), ID, adjusted values, effect size, percent TSA, and day ranges for Drosophila campaigns using clotrimazole, 1-triphenylmethyl imidazole 1-(3-(trifluoromethyl)trityl)imidazole respectively.

TABLE 4

| Compound ID | Conc | Campaign ID | Day Range | Adj Value | Effect Size | se.ES |
|---|---|---|---|---|---|---|
| CLOTRIMAZOLE: | 50 | C00083 | 1 to 7 | 11.20002 | 0.725 | 0.433 |
| CLOTRIMAZOLE: | 50 | C00084 | 1 to 7 | 10.97688 | 0.882 | 0.476 |
| CLOTRIMAZOLE: | 50 | C00085 | 1 to 7 | 10.14642 | 0.932 | 0.354 |
| CLOTRIMAZOLE: | 50 | C00083; | 1 to 7 | | 0.867 | 0.245 |
| | | C00084; C00085 | | | | |
| CLOTRIMAZOLE: | 100 | C00074 | 1 to 7 | 10.84045 | 1.432 | 0.433 |
| CLOTRIMAZOLE: | 100 | C00076 | 1 to 7 | 10.11804 | 0.301 | 0.438 |
| CLOTRIMAZOLE: | 100 | C00083 | 1 to 7 | 11.31555 | 0.962 | 0.433 |
| CLOTRIMAZOLE: | 100 | C00084 | 1 to 7 | 11.4873 | 1.915 | 0.476 |
| CLOTRIMAZOLE: | 100 | C00089 | 1 to 7 | 10.62535 | 0.428 | 0.433 |
| CLOTRIMAZOLE: | 100 | C00074; C00076 | 1 to 7 | | 0.89 | 0.345 |
| | | C00083; | | | | |
| | | C00084; C00089 | | | | |
| CLOTRIMAZOLE: | 150 | C00083 | 1 to 7 | 11.15472 | 0.633 | 0.433 |
| CLOTRIMAZOLE: | 150 | C00084 | 1 to 7 | 11.67982 | 2.304 | 0.5 |
| CLOTRIMAZOLE: | 150 | C00086 | 1 to 7 | 10.57128 | 1.699 | 0.433 |
| CLOTRIMAZOLE: | 150 | C00088 | 1 to 7 | 10.73952 | 0.027 | 0.433 |
| CLOTRIMAZOLE: | 150 | C00089 | 1 to 7 | 11.13356 | 1.667 | 0.5 |
| CLOTRIMAZOLE: | 150 | C00083; | 1 to 7 | | 1.211 | 0.438 |
| | | C00084; C00086 | | | | |
| | | C00088; C00089 | | | | |
| 1-triphenylmethyl imidazole | 100 | C00080 | 1 to 7 | 10.61977 | 0.771 | 0.433 |
| 1-triphenylmethyl imidazole | 100 | C00083 | 1 to 7 | 9.99803 | −0.966 | 0.433 |
| 1-triphenylmethyl imidazole | 100 | C00084 | 1 to 7 | 10.70578 | 0.334 | 0.476 |
| 1-triphenylmethyl imidazole | 100 | C00088 | 1 to 7 | 11.18325 | 0.818 | 0.433 |
| 1-triphenylmethyl imidazole | 100 | C00089 | 1 to 7 | 10.50602 | 0.404 | 0.5 |
| 1-triphenylmethyl imidazole | 100 | C00089 | 1 to 7 | 10.30715 | −0.229 | 0.5 |
| 1-triphenylmethyl imidazole | 100 | C00080; | 1 to 7 | | 0.186 | 0.333 |
| | | C00083; | | | | |
| | | C00084; | | | | |
| | | C00088; C00089 | | | | |
| 1-(3-(TRIFLUOROMETHYL)TRITYL)IMIDAZOLE | 100 | C00083 | 1 to 7 | 10.37422 | −0.293 | 0.433 |
| 1-(3-(TRIFLUOROMETHYL)TRITYL)IMIDAZOLE | 100 | C00084 | 1 to 7 | 10.38315 | −0.319 | 0.476 |
| 1-(3-(TRIFLUOROMETHYL)TRITYL)IMIDAZOLE | 100 | C00089 | 1 to 7 | 10.47412 | 0.116 | 0.5 |
| 1-(3-(TRIFLUOROMETHYL)TRITYL)IMIDAZOLE | 100 | C00083; | 1 to 7 | | −0.159 | 0.299 |
| | | C00084; C00089 | | | | |
| 1-(3-(TRIFLUOROMETHYL)TRITYL)IMIDAZOLE | 300 | C00080 | 1 to 7 | 11.11399 | 1.76 | 0.433 |
| 1-(3-(TRIFLUOROMETHYL)TRITYL)IMIDAZOLE | 300 | C00083 | 1 to 7 | 10.33425 | −0.364 | 0.433 |
| 1-(3-(TRIFLUOROMETHYL)TRITYL)IMIDAZOLE | 300 | C00084 | 1 to 7 | 10.90075 | 0.728 | 0.5 |
| 1-(3-(TRIFLUOROMETHYL)TRITYL)IMIDAZOLE | 300 | C00085 | 1 to 7 | 10.33462 | 1.36 | 0.354 |
| 1-(3-(TRIFLUOROMETHYL)TRITYL)IMIDAZOLE | 300 | C00088 | 1 to 7 | 11.10127 | 0.672 | 0.433 |
| 1-(3-(TRIFLUOROMETHYL)TRITYL)IMIDAZOLE | 300 | C00089 | 1 to 7 | 11.01994 | 1.438 | 0.5 |
| 1-(3-(TRIFLUOROMETHYL)TRITYL)IMIDAZOLE | 300 | C00080; | 1 to 7 | | 1.003 | 0.342 |
| | | C00083; | | | | |
| | | C00084; | | | | |
| | | C00085; | | | | |
| | | C00088; C00089 | | | | |
| 1-[4-(FLUORO)-1,2-TRITYL]IMIDAZOLE | 50 | C00091 | | | 0.31 | |
| 1-[4-(FLUORO)-1,2-TRITYL]IMIDAZOLE | 100 | C00091 | | | 1.04 | |
| 1-[4-(FLUORO)-1,2-TRITYL]IMIDAZOLE | 200 | C00091 | | | 1.23 | |

| Compound ID | Percent TSA | Day Range | Adj Value | Effect Size | Percent TSA |
|---|---|---|---|---|---|
| CLOTRIMAZOLE: | 26.3 | 8 to 10 | 6.586556 | 1.458 | 38.8 |
| CLOTRIMAZOLE: | 25.4 | 8 to 10 | 5.802219 | 1.39 | 31.5 |
| CLOTRIMAZOLE: | 48.8 | 8 to 10 | 6.192592 | 0.767 | 36.8 |
| CLOTRIMAZOLE: | 37.7 | 8 to 10 | | 1.076 | 36.3 |
| CLOTRIMAZOLE: | 54.7 | 8 to 10 | 5.805369 | 1.319 | 40.9 |
| CLOTRIMAZOLE: | 15.5 | 8 to 10 | 5.533756 | 0.385 | 22.3 |
| CLOTRIMAZOLE: | 34.9 | 8 to 10 | 6.208165 | 0.849 | 22.6 |
| CLOTRIMAZOLE: | 55.2 | 8 to 10 | 6.980823 | 3.682 | 83.5 |
| CLOTRIMAZOLE: | 17.2 | 8 to 10 | 6.154569 | 1.286 | 31.7 |
| CLOTRIMAZOLE: | 32 | 8 to 10 | | 1.372 | 36.8 |
| CLOTRIMAZOLE: | 23 | 8 to 10 | 6.188744 | 0.817 | 21.8 |
| CLOTRIMAZOLE: | 66.5 | 8 to 10 | 6.817065 | 3.363 | 76.2 |
| CLOTRIMAZOLE: | 67.4 | 8 to 10 | 5.85489 | 1.662 | 43.5 |
| CLOTRIMAZOLE: | 1.3 | 8 to 10 | 5.982558 | 1.237 | 33.8 |
| CLOTRIMAZOLE: | 66.3 | 8 to 10 | 6.595031 | 1.879 | 51.4 |
| CLOTRIMAZOLE: | 43.8 | 8 to 10 | | 1.664 | 42.8 |
| 1-triphenylmethyl imidazole | 31 | 8 to 10 | 5.546526 | 0.617 | 16.8 |
| 1-triphenylmethyl imidazole | −29.7 | 8 to 10 | 5.810717 | 0.856 | 37.3 |
| 1-triphenylmethyl imidazole | 9.6 | 8 to 10 | 4.931636 | −0.897 | −22.8 |
| 1-triphenylmethyl imidazole | 39.6 | 8 to 10 | 5.407234 | 0.621 | 14.1 |
| 1-triphenylmethyl imidazole | 16.1 | 8 to 10 | 5.09158 | −0.74 | −20.3 |
| 1-triphenylmethyl imidazole | −9.2 | 8 to 10 | 5.877167 | 0.727 | 17.9 |
| 1-triphenylmethyl imidazole | 9.6 | 8 to 10 | | 0.179 | 6.9 |
| 1-(3-(TRIFLUOROMETHYL)TRITYL)IMIDAZOLE | −9 | 8 to 10 | 5.093991 | −0.651 | −16.5 |
| 1-(3-(TRIFLUOROMETHYL)TRITYL)IMIDAZOLE | −9.2 | 8 to 10 | 5.776492 | 1.339 | 30.4 |
| 1-(3-(TRIFLUOROMETHYL)TRITYL)IMIDAZOLE | 4.6 | 8 to 10 | 5.81051 | 0.592 | 14.6 |
| 1-(3-(TRIFLUOROMETHYL)TRITYL)IMIDAZOLE | −4.3 | 8 to 10 | | 0.344 | 7.6 |
| 1-(3-(TRIFLUOROMETHYL)TRITYL)IMIDAZOLE | 70.8 | 8 to 10 | 7.096252 | 2.877 | 125.3 |
| 1-(3-(TRIFLUOROMETHYL)TRITYL)IMIDAZOLE | −11.2 | 8 to 10 | 5.023627 | −0.757 | −19.2 |
| 1-(3-(TRIFLUOROMETHYL)TRITYL)IMIDAZOLE | 21 | 8 to 10 | 5.921476 | 1.621 | 36.7 |
| 1-(3-(TRIFLUOROMETHYL)TRITYL)IMIDAZOLE | 71.3 | 8 to 10 | 7.137214 | 1.961 | 94 |
| 1-(3-(TRIFLUOROMETHYL)TRITYL)IMIDAZOLE | 32.6 | 8 to 10 | 6.975946 | 2.65 | 72.3 |
| 1-(3-(TRIFLUOROMETHYL)TRITYL)IMIDAZOLE | 57.2 | 8 to 10 | 6.600783 | 1.889 | 51.7 |
| 1-(3-(TRIFLUOROMETHYL)TRITYL)IMIDAZOLE | 45.6 | 8 to 10 | | 1.798 | 68.2 |
| 1-[4-(FLUORO)-1,2-TRITYL]IMIDAZOLE | 16.8 | | | 0.33 | 12.5 |
| 1-[4-(FLUORO)-1,2-TRITYL]IMIDAZOLE | 56.8 | | | 1.32 | 49.9 |
| 1-[4-(FLUORO)-1,2-TRITYL]IMIDAZOLE | 67.2 | | | 0.8 | 30.2 |

### Example 4

### Mouse Model of Acute Neurodegeneration: Kainic Acid Lesion

Poly (ADP-ribose) polymerase (PARP) is a DNA binding protein that uses NAD+ as substrate. PARP is activated by strand breaks in the DNA molecule that can be induced by DNA damaging agents, including free radicals (Murcia et al., 1994 [19]). When fully activated, for example by free radical-induced DNA damage, PARP can deplete cellular energy stores, under the form of NAD+ and ATP, predisposing the cell to death (Berger, 1985 [20]). In vitro and in vivo studies using PARP inhibitors, including benzamide, and/or mice or cells with a disrupted PARP-1 gene, have indicated a participation of PARP in glutamate- and N-methyl-D-aspartate (NMDA)-induced neurotoxicities, cerebral ischemia, and 1-methyl-4-phenyl-1,2,3,6-tetrahydropyridine (MPTP), and methamphetamine-induced neurotoxicities (Cosi et al., 2004 [21]). In particular, it has been shown that the PARP inhibitor benzamide is neuroprotective in C57B1/6N mice against different types of neurotoxicities. Kainic acid-induced (KA) neurotoxicity is related to free radical formation via membrane lipid peroxidation and the arachidonic acid cascade and ATP depletion. In particular, KA injected into the striatum of mice causes a rapid decline in striatal levels of ATP (Retz and Coyle, 1980 [22]), and nanomolar amounts of KA injected into the striatum of the rat can produce time dependent changes in striatal PARP activity (Cosi et al., 2000 [23]).

Cosi et al., investigated the time-course of KA-induced toxicity and the effects of the PARP inhibitor, benzamide, on KA neurotoxicities in vivo, by measuring changes in the volume of the lesion induced by the intra-striatal (i.s.) injection of these excitotoxins in C57B1/6N mice (Cosi et al., 2004 [21]). The KA-induced lesion volume was dependent on the amount of toxin injected and the survival time. KA also produced an extensive astrogliosis. Benzamide partially prevented KA-induced lesions and astrogliosis. The effects of benzamide appeared to be in part related to changes in energy metabolism, since KA produced decreases in striatal levels of NAD+ and ATP that were partially prevented by benzamide. These results indicate that PARP overactivation and energy depletion could be responsible in part for the cellular demise during the development of the lesion induced by KA.

By extension, it is possible that activation of PARP in KA treated mice leads to elevated levels of intracellular ADP-ribose and oxidative stress which potentially culminates in the activation of TRPM-2. Over-activation of TRPM-2 would then flood the cell with Ca²⁺ predisposing the cell to death. Clotrimazole has been shown to be a potent inhibitor of microglia activation so it may also help in moderating the neuroinflamation/astrogliosis caused KA.

To test the hypothesis that clotrimazole may be neuroprotective against KA induced excitatory damage in the brain, clotrimazole was compared to benzamide as a positive control in the mouse model. Three study groups (n=8-9) were tested: (1) C57BL/6 treated with KA 1.0 nmol (i.s.) left hemisphere+vehicle (i.p.); (2) KA 1.0 nmol i.s. left hemisphere+clotrimazole (2mg/kg) i.p. twice daily for duration of the study (including a single dose 1 hour before KA); and (3) KA 1.0 nmol i.s. left hemisphere+benzamide (160 mg/kg, i.p. 30 min before injection and 3.5 hrs after). Kainic Acid (K0250), clotrimazole (C6019) and Benzamide (150762) were purchased from Sigma and prepared as followed: Kainic Acid was dissolved in phosphate buffered saline (1×PBS, pH 7.4), pH to 7.4 using NaOH. Benzamide was prepared in 0.9% NaCl containing 0.01% methylcellulose (32mg benzamide/mL vehicle) and clotrimazole in a PEG400:Cremophor EL:Water (10:10:80) at pH 6.8. Clotrimazole or vehicle (100 ul i.p.) was dosed twice daily (B.I.D.) for 1 week before KA challenge.

Two days after injection of KA the animals were anesthetized and transcardially perfused with ice cold phosphate buffered saline (0.1 M PBS, pH 7.4) containing 10% sucrose and then perfused with ice-cold 4% paraformaldehyde in 0.1M PBS, pH 7.4. Heads were transferred into ice cold 4% paraformaldehyde overnight at 4° C. At least 24 hours later the brains were extracted from the skull and transferred back into 0.1M PBS prior to embedding. All brains were embedded together in a solid matrix and sectioned coronally as a single unit, starting from the rostral limit of the striatum, into ˜230 sequential rostrocaudal sections of 35μm thickness. Embedding, sectioning and processing all the brains together provides uniformity of treatment and staining across treatment groups. These sections encompassed both the entire span of the lesioned tissue and the entire striatum and cortex. One out of every 6 sections was mounted on gelatinized slides dehydrated then rehydrated and stained thionine (Nissl method). The area of the lesion, indicated by the lack of staining when compared to the contralateral (intact) striatum/cortex, was measured in each series of sections (n=25-30) blindly by means of an image analysis program and the lesion volume was calculated by a stereological method (Cavalieri's estimator of volume corrected for over projection).

Clotrimazole demonstrated statistically significant improvement in lesion volume (P=0.010) for KA+clotrimazole compared to KA+vehicle alone (t test of difference in log [size]).

### Example 5

### Transgenic Mouse Model of Huntington's Disease

One of the most widely studied Huntington's Disease (HD) murine models is the R6/2 transgenic mouse. Extensive behavioral and neuropathological studies have provided a foundation for the use of R6/2 mice in preclinical therapeutic trials (Beal and Ferrante, 2004 [24]). The R6/2 model has many of the temporal, behavioral, and neuropathological features that are observed in patients with HD, such as motor dysfunction and striatal atrophy. Neuropathological outcome measures include gross and cellular striatal atrophy as well as numbers of protein aggregates/inclusions detected with anti-Huntington antibodies. One advantage of using R6/2 mice is that it is possible to perform survival studies in less than three months. The effect of a treatment on this endpoint has been used as a relevant surrogate for neuroprotection. It also correlates well with improvements in motor performance, which is assessed as performance on the rotarod test and as maintenance of body weight, both of which are impaired in HD (Mangiarini et al., 1996 [25]; Stack et al., 2005 [26]).

The rotarod test and body weight measurements were used to assess the therapeutic effects of clotrimazole in R6/2 transgenic mice. R6/2 and WT male mice at 4 weeks of age were trained 3 consecutive days on the accelerating rotarod. Each training day consisted of four sessions. After the training was complete, the animals were reassigned to new groups to create statistically homogenous cohorts. At 5 weeks of age, animals (n=10 per group) were dosed twice daily, intraperitoneally (IP) with clotrimazole at either 2 mg/kg or 10 mg/kg (equal to 4 and 20 mg/kg per day). Rotarod testing was performed at 6, 8, 10, and 12 weeks of age as described above. Body weights were collected every week.

Preliminary data suggests clotrimazole at the low dose of 4 mg/kg/day positively affects behavior and weight in vivo.

A statistically-significant effect was seen at the higher dse of clotrimaole (P=0.01 vs. vehicle-treated R6/2 animals (Wilcoxon test)). The 20 mg/kg/day IP-BID dose did not have a statistically significant effect at any time point tested.

An improvement was seen in the R6/2 mice dosed at 4 mg/kg by week 12. R6/2 mice dosed at 4 mg/kg clotrimazole IP-BID at 12 week of age maintained a higher body weight compared to vehicle-treated R6/2 mice (F test ANOVA). The 20 mg/kg IP-BID dose did not have a statistically significant effect at any time point tested.

Without wishing to be bound by any particular theory, the U-shaped dose response of CLT may be due to an increased level of “off target” effects for CLT at higher concentrations which lessen therapeutic effect of CLT that occurs at lower concentrations. Known side affects in humans include lethargy, apathy, and weakness, all of which could manifest as poor rotarod performance in mice.

### Example 6

### Cage Climbing and Gait Analysis in Mouse Model of HD

Cage climbing, a general descriptor of motor activity and dexterity, was described to be a naturally occurring activity that has been used to highlight significant behavioral differences between wild-type (WT) and R6/2 mice (Hickey et al., 2005 [27]). As the mice age, the disparity between WT and TG mice grows considerably.

Cage climbing analysis involves filming a 10cm tall cylindrical cage into which an R6/2 (+/−drug) or WT mouse is placed. The mouse is allowed to move naturally ad libetum for a five minute session within the confines of the cage, while events such as rearing occurrences, climbing time and latency to climb are documented. Cage climbing and rearing data were collected at 12 weeks in this study.

Gait analysis was also collected at 12 weeks, which is a time when clear differences have previously been described between R6/2 and wild-type. In this analysis, the DigiGait™ Imaging System, which is non-invasive, robust, and quantitative—in that it generates numerous indices of gait dynamics and posture—was used. The system simplifies kinematic observations and analyses by imaging the animals from below a transparent treadmill. Software, including artificial intelligence algorithms, quantifies the characteristics of gait, including step sequence patterns, stride length, cadence, and paw placement. The output also includes swing and stance durations, as well as braking and propulsion durations. Indices computed by DigiGait convey information about sensory and motor inputs modulating gait and gait variability.

Preliminary data suggests clotrimazole at the 4 and 20 mg/kg dosing significantly improved the motor performance as assayed by (a) cage climbing and rearing and (b) gait analysis.

Statistically significant improvement was seen in the R6/2 mice dosed with clotrimazole at 4 mg/kg (low dose) and 20 mg/kg (high dose) for cage climbing at 12 weeks In addition, Clotrimazole had a significant effect in the gait analysis at 12 weeks by Dunnett's. Clotrimazole significantly improved the swing ratio score. As the metrics are not mutually exclusive (e.g. stance, braking, etc.), improvements with clotrimazole were also noted with in other scores.

### REFERENCES

1. Leegwater-Kim J, Jang-Ho C. The paradigm of Huntington's disease: therapeutic opportunities in neurodegeneration. NeuroRx. 2004; 1(1):128-138.
2. Gardian G, Vecsei L. Huntington's Disease: pathomechanism and therapeutic perspectives. J Neural Transm. 2004; 111:1485-1494.
3. Hague S M, Klaffke S, Bandmann O. Neurodegenerative disorders: Parkinson's disease and Huntington's disease. J Neurol Neurosurg Psychiatry. 2005; 76:1058-1063.
4. Grimbergen Y A M, Roos R A C. Therapeutic options for Huntington's disease. Curr Opin Invest Drugs. 2003; 4(1):51-54.
5. Isaev N K, Stelmashook E V, Dimagl U, Andreeva A, Manuhova L, Vorobjev V S, et al. Neuroprotective effects of the antifungal drug clotrimazole. Neuroscience. 2002; 113(1):47-53.
6. Eder P, Poteser M, Romanin C. Na⁺ entry and modulation of Na⁺/Ca⁺ exchange as a key mechanism of TRPC signaling. Pflugers Arch—Eur J Physiol. 2005; 451:99-104.
7. Khanna R, Roy L, Zhu X, Schlichter L C. K⁺ channels and the microglial respiratory burst. Am J Physiol Cell Physiol. 2001; 280:C796-C801.
8. Hill K, McNulty S, Randall A D. Inhibition of TRPM2 channels by the antifungal agents clotrimazole and econazole. Naunyn-Schmiedeberg's Arch Pharmacol. 2004; 370:227-237.
9. Hill K, Tigue N J, Kelsell R E, Benham C D, McNulty S, Schaefer M, et al. Characterization of recombinant rat TRPM2 and TRPM2-like conductance in cultured rat striatal neurones. Neuropharm. 2005; In-press.
10. Aktas H, Flückiger R, Acosta J A, Savage J M, Palakurthi S S, Halperin J A. Depletion of intracellular Ca²⁺ stores, phosphorylation of eIF2α, and sustained inhibition of translation initiation mediate the anticancer effects of clotrimazole. Proc Natl Acad Sci USA. 1998; 95:8280-8285.
11. Bauer M K A, Vogt M, Los M, Siegel J, Wesselborg S, Schulze-Osthoff K. Role of reactive oxygen intermediates in activation-induced CD95 (APO-1/Fas) ligand expression. J Biol Chem. 1998; 273(14):8048-8055.
12. Khalid M H, Shibata S, Hiura T. Effects of clotrimazole on the growth, morphological characteristics, and cisplatin sensitivity of human glioblastoma cells in vitro. J Neurosurg. 1999; 90:918-927.
13. Khalid M H, Tokunaga Y, Caputy A J, Walters E. Inhibition of tumor growth and prolonged survival of rats with intracranial gliomas following administration of clotrimazole. J Neurosurg. 2005; 103:79-86.
14. Sun J, Sui X X, Bradbury J A, Zeldin D C Conte M S, Liao J K. Inhibition of vascular smooth muscle cell migration by cytochrome P450 epoxygenase-derived eicosanoids. Circ Res. 2002; 90:1020-1027.
15. Wojtulewski J A, Gow P J, Walter J, Grahame R, Gibson T, Panayi G S, et al. Clotrimazole in rheumatoid arthritis. Annals of the Rheumatic Diseases. 1980; 39:469-472.
16. Protocol: Study of clotrimazole and hydroxyurea in patients with sickle cell syndrome. ClinicalTrials.gov Identifier: NCT00004404. 1997; Ongoing.
17. Protocol: Phase I/II randomized study of hydroxyurea with or without clotrimazole in patients with sickle cell anemia. ClinicalTrials.gov Identifier: NCT00004492. 1999; Ongoing.
18. Protocol: Clotrimazole enemas for pouchitis in children and adults. ClinicalTrials.gov Identifier: NCT00061282. 2002; Ongoing.
19. de Murcia G, Schreiber V, Molinete M, Saulier B, Poch O, Masson M, et al. Structure and function of poly(ADP-ribose) polymerase. Mol Cell Biochem. 1994, 138:15-24.
20. Berger, N A. Poly(ADP-ribose) in the cellular response to DNA damage. Radiat. Res. 1985, 101:4-15.
21. Cosi C, Guerin K, Marien M, Koek W, Rollet K. The PARP inhibitor benzamide protects against kainate and NMDA but not AMPA lesioning of the mouse striatum in vivo. Brain Res. 2004, 1:1-8.
22. Retz K C, Coyle J T. Kainic acid lesion of mouse striatum: effects on energy metabolites. Life Sci. 1980, 25-26:2495-500.
23. C. Cosi, E. Cavalieri, A. Carcereri de Prati, M. Marien, H. Suzuki. Effects of kainic acid lesioning on poly(ADP-ribose) polymerase (PARP) activity in the rat striatum in vivo. Amino Acids. 2000, 19:229-237.
24. Beal M F, Ferrante R J. Experimental therapeutics in transgenic mouse models of Huntington's disease. Nat Rev Neurosci. 2004, 5:373-84.
25. Mangiarini L, Sathasivam K, Seller M, Cozens B, Harper A, Hetherington C, et al. Exon 1 of the HD gene with an expanded CAG repeat is sufficient to cause a progressive neurological phenotype in transgenic mice. Cell. 1996, 87:493-506.
26. Stack E C, Kubilus J K, Smith K, Cormier K, Del Signore S J, Guelin E, et al. Chronology of behavioral symptoms and neuropathological sequela in R6/2 Huntington's disease transgenic mice. J Comp Neurol. 2005; 490:354-370.
27. Hickey M A, Gallant K, Gross G G, Levine M S, Chesselet M F. Early behavioral deficits in R6/2 mice suitable for use in preclinical drug testing. Neurobiol Dis. 2005, 20:1-11.

All patents, patent applications, and published references cited herein are hereby incorporated by reference in their entirety. While this invention has been particularly illustrated and described with reference to particular examples, it will be understood by those skilled in the art that various changes in form and details may be made therein without departing from the scope and spirit of the invention encompassed by the appended claims.

What is claimed is:

1. A method of treating a subject having a neurological disorder, comprising administering to said subject an effective amount of a compound of the Formula (I) or a pharmaceutically acceptable salt thereof 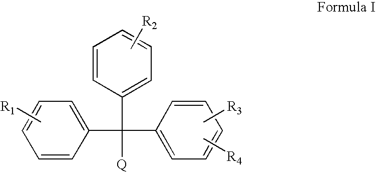
wherein R₁, R₂, R₃ and R₄ are independently selected from the group consisting of a hydrogen, halogen, cyano, trifluoromethyl, carboxylic acid (CO₂H), carboxamide (CON(R₅)₂), nitro, hydroxyl, alkoxy, mercapto, alkylthio, alkylsulfonyl, amino, alkylamino, dialkylamino, acylamino, aryl, heteroaryl, alkenyl, alkynyl, cycloalkyl, heterocycloalkyl, alkyl and substituted alkyl;
wherein each R₅ is independently selected from the group consisting of a hydrogen, cycloalkyl, alkyl and substituted alkyl; and
wherein Q is selected from the group consisting of a hydrogen, hydroxyl, alkoxy, alkylthio, alkylamino, dialkylamino, acylamino or a heterocyclic group.

2. The method of claim 1, wherein the heterocyclic group is selected from the group consisting of N-morpholino, 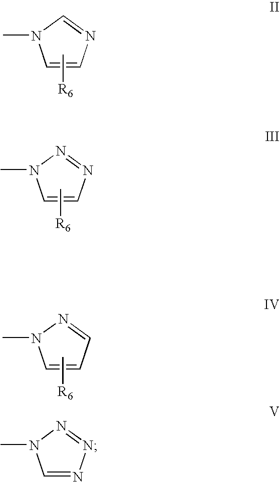
wherein R₆ is selected from the group consisting of a hydrogen, halogen, nitro, cyano, alkyl, alkoxy, and CON(R₅)₂.

3. The method of claim 1, wherein said neurological disorder is a neurodegenerative disease.

4. The method of claim 1, wherein said neurological disorder is a disorder of movement.

5. The method of claim 1, wherein said neurological disorder is an extrapyramidal disorder or a cerebellar disorder.

6. The method of claim 1, wherein said neurological disorder is a hyperkinetic movement disorder.

7. The method of claim 1, wherein said neurological disorder is selected from the group consisting of Alzheimer's disease, Huntington's disease, Parkinson's disease, age-related memory impairment, amyotrophic lateral sclerosis, ataxia-telangiectasia, Biswanger's disease, cerebral amyloid angiopathies, Creutzfeldt-Jacob disease including variant form, corticobasal degeneration, multi infarct dementia, subcortical dementia, dementia with Lewy Bodies, dementia due to human immunodeficiency virus (HIV), Friedreich ataxia, fronto-temporal dementia linked to chromosome 17 (FTDP-17), frontotemporal lobar degeneration, frontal lobe dementia, Kennedy disease, Korsakoff's syndrome, mild cognitive impairment, neurological manifestations of HIV, neurological conditions arising from polyglutamine expansions, Pick's disease, prion diseases, Kuru disease, fatal familial insomnia, Gerstmann-Straussler-Scheinker disease, prion protein cerebral amyloid angiopathy, postencephalitic Parkinsonism, progressive supemuclear palsy, Rett syndrome, spinal muscular atrophy, transmissable spongiform encephalopathies and vascular dementia.

8. The method of claim 7, wherein said neurological disorder is selected from the group consisting of Alzheimer's disease, Huntington's disease, Parkinson's disease and a neurological condition arising from a polyglutamine expansion.

9. The method of claim 8, wherein said neurological disease is a neurological condition arising from a polyglutamine expansion.

10. The method of claim 9, wherein said polyglutamine expansion is of at least 10 residues.

11. The method of claim 9, wherein said polyglutamine expansion is of at least 20 residues.

12. The method of claim 9, wherein said polyglutamine expansion is between 21 and 33 residues in length.

13. The method of claim 8, wherein said neurological disorder is Huntington's disease.

14. The method of claim 1, wherein said compound of the Formula (I) or a pharmaceutically acceptable salt thereof is administered in combination with at least one additional active agent.

15. The method of claim 14, wherein said additional active agent is selected from the group consisting of tiapride; pimozide; haloperidol; tetrabenazine; phenothiazines; an antiparkinsonian medication, such as levodopa, dopamine agonists, and anticholinergics; tricyclic antidepressants; SSRIs, monoamine oxidase inhibitors; benzodiazepines; amitriptyline; antipsychotics; propranolol; pindolo; classical antipsychotics; and clozapine.

16. The method of claim 1, wherein said compound of the Formula (I) or a pharmaceutically acceptable salt thereof is administered as a pharmaceutical composition further comprising at least one excipient, carrier or diluent.

17. The method of claim 16, wherein said pharmaceutical composition is administered in a solid dosage form or in a liquid dosage form.

18. The method of claim 17, wherein said dosage form is selected from the group consisting of an oral dosage form, a parenteral dosage form, an intranasal dosage form, a suppository, a lozenge, a troche, buccal, a controlled release dosage form, a pulsed release dosage form, an immediate release dosage form, an intravenous solution, a suspension and combinations thereof.

19. The method of claim 18, wherein said dosage form is an oral dosage form.

20. The method of claim 19, wherein said oral dosage form is a controlled release dosage form.

21. The method of claim 19, wherein said oral dosage form is a tablet, capsule or a caplet.

22. The method of claim 16, wherein said pharmaceutical composition is administered using a shunt.

23. The method of claim 1, wherein said subject is a mammal.

24. The method of claim 23, wherein said mammal is a human.

25. The method of claim 1, wherein said compound of the Formula (I) is: 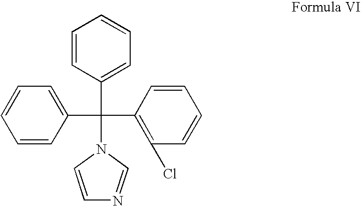
or a pharmaceutically acceptable salt thereof.

26. The method of claim 1, wherein said compound of the Formula (I) is: 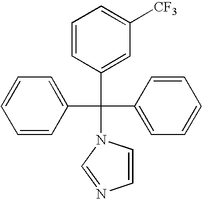
or a pharmaceutically acceptable salt thereof.

27. The method of claim 1, wherein said compound of the Formula (I) is: 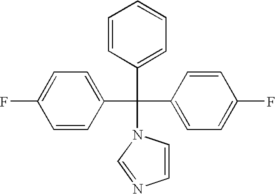
or a pharmaceutically acceptable salt thereof.

28. The method of claim 1, wherein said compound of the Formula (I) is: 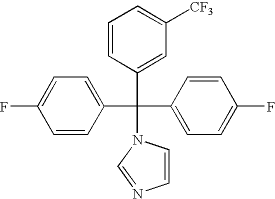
or a pharmaceutically acceptable salt thereof.

29. A pharmaceutical composition for treating a subject having a neurological disorder, said pharmaceutical composition comprising an effective amount of a compound of the Formula (I) or a pharmaceutically acceptable salt thereof, and a pharmaceutically acceptable excipient, carrier or diluent.

30. The pharmaceutical composition of claim 29, wherein said compound of the Formula (I) is: 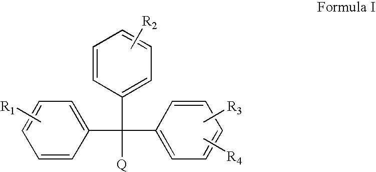
wherein R₁, R₂, R₃ and R₄ are independently selected from the group consisting of a hydrogen, halogen, cyano, trifluoromethyl, carboxylic acid (CO₂H), carboxamide (CON(R₅)₂), nitro, hydroxyl, alkoxy, mercapto, alkylthio, alkylsulfonyl, amino, alkylamino, dialkylamino, acylamino, aryl, heteroaryl, alkenyl, alkynyl, cycloalkyl, heterocycloalkyl, alkyl and substituted alkyl;
wherein each R₅ is independently selected from the group consisting of a hydrogen, cycloalkyl, alkyl and substituted alkyl; and
wherein Q is selected from the group consisting of a hydrogen, hydroxyl, alkoxy, alkylthio, alkylamino, dialkylamino, acylamino or heterocyclic group.

31. The pharmaceutical composition of claim 30, wherein the heterocyclic group is selected from the group consisting of N-morpholino, 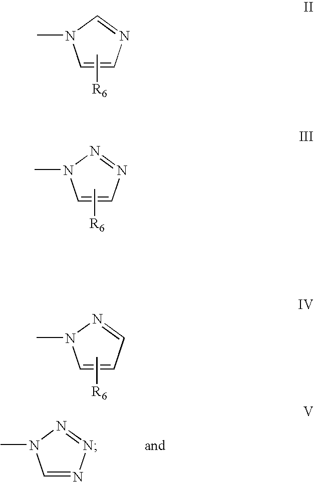
wherein R₆ is selected from the group consisting of a hydrogen, halogen, nitro, cyano, alkyl, alkoxy, and CON(R₅)₂;
or a pharmaceutically acceptable salt thereof.

32. The pharmaceutical composition of claim 30, wherein said compound of the Formula (I) is: 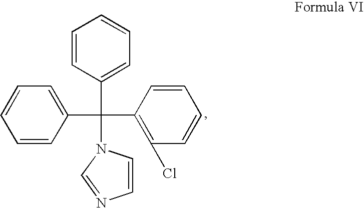
or a pharmaceutically acceptable salt thereof.

33. The pharmaceutical composition of claim 30, wherein said compound of the Formula (I) is: 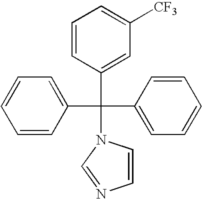
or a pharmaceutically acceptable salt thereof.

34. The pharmaceutical composition of claim 30, wherein said compound of the Formula (I) is: 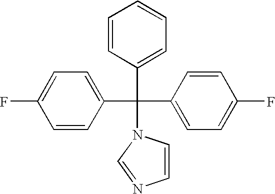
or a pharmaceutically acceptable salt thereof.

35. The pharmaceutical composition of claim 30, wherein said compound of the Formula (I) is: 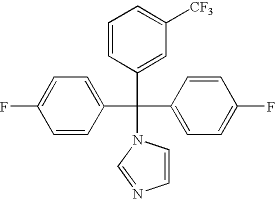
or a pharmaceutically acceptable salt thereof.

36. The method of claim 25, wherein said neurological disorder is a neurodegenerative disease.

37. The method of claim 25, wherein said neurological disorder is a disorder of movement.

38. The method of claim 25, wherein said neurological disorder is an extrapyramidal disorder or a cerebellar disorder.

39. The method of claim 25, wherein said neurological disorder is a hyperkinetic movement disorder.

40. The method of claim 25, wherein said neurological disorder is selected from the group consisting of Alzheimer's disease, Huntington's disease, Parkinson's disease, age-related memory impairment, amyotrophic lateral sclerosis, ataxia-telangiectasia, Biswanger's disease, cerebral amyloid angiopathies, Creutzfeldt-Jacob disease including variant form, corticobasal degeneration, multi infarct dementia, subcortical dementia, dementia with Lewy Bodies, dementia due to human immunodeficiency virus (HIV), Friedreich ataxia, fronto-temporal dementia linked to chromosome 17 (FTDP-17), frontotemporal lobar degeneration, frontal lobe dementia, Kennedy disease, Korsakoff's syndrome, mild cognitive impairment, neurological manifestations of HIV, neurological conditions arising from polyglutamine expansions, Pick's disease, prion diseases, Kuru disease, fatal familial insomnia, Gerstmann-Straussler-Scheinker disease, prion protein cerebral amyloid angiopathy, postencephalitic Parkinsonism, progressive supernuclear palsy, Rett syndrome, spinal muscular atrophy, transmissable spongiform encephalopathies and vascular dementia.

41. The method of claim 40, wherein said neurological disorder is selected from the group consisting of Alzheimer's disease, Huntington's disease, Parkinson's disease and a neurological condition arising from a polyglutamine expansion.

42. The method of claim 41, wherein said neurological disease is a neurological condition arising from a polyglutamine expansion.

43. A compound represented by the formula: 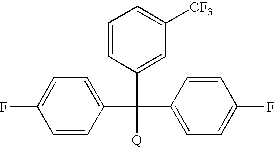
wherein Q is selected from the group consisting of a hydrogen, hydroxyl, alkoxy, alkylthio, alkylamino, dialkylamino, acylamino or a heterocyclic group;
or a pharmaceutically acceptable salt thereof.

44. The compound of claim 43, wherein the heterocyclic group is selected from the group consisting of N-morpholino, 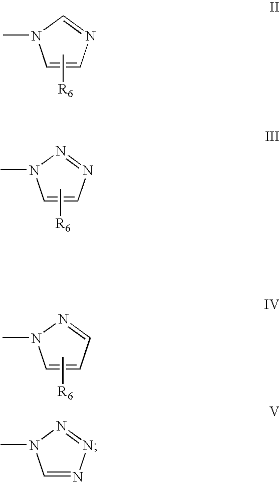
wherein R₆ is selected from the group consisting of a hydrogen, halogen, nitro, cyano, alkyl, alkoxy, and CON(R₅)₂; and
wherein each R₅ is independently selected from the group consisting of a hydrogen, cycloalkyl, alkyl and substituted alkyl.

45. The compound of claim 44, wherein the compound is represented by the structure: 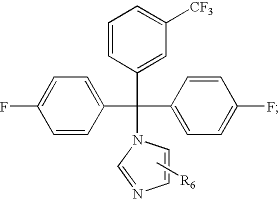
or a pharmaceutically acceptable salt thereof.

46. The compound of claim 45, wherein the compound is: 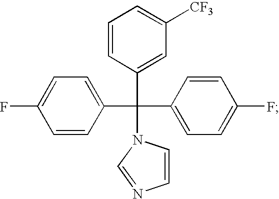
or a pharmaceutically acceptable salt thereof.

47. A pharmaceutical composition comprising a compound of claim 43, or a pharmaceutically acceptable salt thereof, and a pharmaceutically acceptable excipient, carrier or diluent.

48. A method of treating a subject having a neurological disorder, comprising administering to said subject an effective amount of a compound of claim 43, or a pharmaceutically acceptable salt thereof.
